SEQUENCE LISTING (11 sequences)

SEQ ID NO: 1 - DNA, 23 bp, Artificial Sequence
  Description of Artificial Sequence Synthetic probe
ttctgacctgaaggctctgcgcg

SEQ ID NO: 2 - DNA, 21 bp, Artificial Sequence
  Description of Artificial Sequence Synthetic probe
  Description of Combined DNA/RNA Molecule Synthetic probe
  modified_base (4)..(4): Locked nucleic acid
  modified_base (11)..(12): Locked nucleic acid
  modified_base (14)..(14): Locked nucleic acid
  modified_base (16)..(16): Locked nucleic acid
ttacagggtttcagacaaaat

SEQ ID NO: 3 - DNA, 22 bp, Artificial Sequence
  Description of Artificial Sequence Synthetic probe
  Description of Combined DNA/RNA Molecule Synthetic probe
  modified_base (5)..(5): Locked nucleic acid
  modified_base (7)..(7): Locked nucleic acid
  modified_base (12)..(13): Locked nucleic acid
  modified_base (15)..(15): Locked nucleic acid
  modified_base (17)..(17): Locked nucleic acid
cttacagggttttagacaaaat

SEQ ID NO: 4 - DNA, 23 bp, Artificial Sequence
  Description of Artificial Sequence Synthetic probe
  Description of Combined DNA/RNA Molecule Synthetic probe
  modified_base (6)..(6): Locked nucleic acid
  modified_base (8)..(8): Locked nucleic acid
  modified_base (10)..(11): Locked nucleic acid
  modified_base (15)..(15): Locked nucleic acid
  modified_base (21)..(21): Locked nucleic acid
ctggactctattttgaaaaacca

SEQ ID NO: 5 - DNA, 21 bp, Artificial Sequence
  Description of Artificial Sequence Synthetic probe
  Description of Combined DNA/RNA Molecule Synthetic probe
  modified_base (6)..(6): Locked nucleic acid
  modified_base (8)..(8): Locked nucleic acid
  modified_base (10)..(10): Locked nucleic acid
  modified_base (13)..(13): Locked nucleic acid
  modified_base (19)..(19): Locked nucleic acid
ctggactcttttgaaaaacca

SEQ ID NO: 6 - DNA, 21 bp, Artificial Sequence
  Description of Artificial Sequence Synthetic primer
gcggtgtttgcagatttggac

SEQ ID NO: 7 - DNA, 18 bp, Artificial Sequence
  Description of Artificial Sequence Synthetic primer
ctccggagtstggcccga

SEQ ID NO: 8 - DNA, 35 bp, Artificial Sequence
  Description of Artificial Sequence Synthetic primer
aatgctttttaacatccatataaagctatctatat

SEQ ID NO: 9 - DNA, 28 bp, Artificial Sequence
  Description of Artificial Sequence Synthetic primer
tgctggcagacttactcyttaatttaag

SEQ ID NO: 10 - DNA, 32 bp, Artificial Sequence
  Description of Artificial Sequence Synthetic primer
attaaaagttatgtaataaccaratgcaatgt

SEQ ID NO: 11 - DNA, 35 bp, Artificial Sequence
  Description of Artificial Sequence Synthetic primer
atccaatatcattcaaaatctaatccacattcaaa

(19) United States
(12) Patent Application Publication
(10) Pub. No.: US 2022/0033884 A1
(43) Pub. Date: Feb. 3, 2022

(54) METHODS AND SYSTEMS FOR IDENTIFICATION OF SPINAL MUSCULAR ATROPHY

(71) Applicants: Robert Daber, (US); Coyote Bioscience USA Inc., Alameda, CA (US)

(72) Inventors: Robert Daber, Farmingdale, NJ (US); Roger Schaller, Morgan Hill, CA (US); Jesus Ching, Saratoga, CA (US)

(21) Appl. No.: 17/370,664

(22) Filed: Jul. 8, 2021

Related U.S. Application Data
(63) Continuation of application No. PCT/US20/12773, filed on Jan. 8, 2020.
(60) Provisional application No. 62/790,430, filed on Jan. 9, 2019.

(51) Int. Cl. C12Q 1/6827; C12Q 1/6858

(52) U.S. Cl. CPC C12Q 1/6827; C12Q 1/6858

(57) ABSTRACT

The present disclosure provides kits, methods and systems for identifying spinal muscular atrophy (SMA) in a subject or identifying the subject as a carrier of SMA.

### CROSS-REFERENCE

This application is a continuation application of International Application No. PCT/US20/12773, filed on Jan. 8, 2020, which claims the benefit of U.S. Provisional Application No. 62/790,430, filed on Jan. 9, 2019, which application is incorporated herein by reference in its entirety.

### SEQUENCE LISTING

The instant application contains a Sequence Listing which has been submitted electronically in ASCII format and is hereby incorporated by reference in its entirety. Said ASCII copy, created on Sep. 27, 2021, is named 45769-751_301_SL.txt and is 4,559 bytes in size.

### BACKGROUND

Nucleic acid amplification methods permit selected amplification and identification of nucleic acids of interest from a complex mixture, such as a biological sample. To detect a nucleic acid in a biological sample, the biological sample is typically processed to isolate nucleic acids from other components of the biological sample and other agents that may interfere with the nucleic acid and/or amplification. Following isolation of the nucleic acid of interest from the biological sample, the nucleic acid of interest can be amplified, via, for example, amplification methods, such as thermal cycling based approaches (e.g., polymerase chain reaction (PCR)). Following amplification of the nucleic acid of interest, the products of amplification can be detected and the results of detection interpreted by an end-user.

### SUMMARY

The present disclosure provides methods and systems for efficient amplification of nucleic acids, such as RNA and DNA molecules. Amplified nucleic acid product can be detected rapidly and with good sensitivity allowing detection of gene markers or copy number for the detection of genetic conditions. Moreover, methods and systems described herein can be implemented in the context of identifying, detecting, diagnosing, treating and/or managing spinal muscular atrophy (SMA) as well as identifying a genetic signature(s) in unaffected individuals who have an increased risk of having offspring affected with SMA. Kits, methods and systems of the present disclosure may identify markers indicative of SMA, but in some instances may by themselves not yield a diagnosis (e.g., a doctor may use reports generated by methods or systems of the present disclosure to render a diagnosis). SMA is a neuromuscular disorder that may cause motor impairment related to a loss of function of motor neurons and can affect the muscles throughout the body, including the limbs or the respiratory system.

In an aspect, the disclosure provides a method for identifying a genetic signature(s) associated with spinal muscular atrophy (SMA) in a nucleic acid sample of a subject, comprising: (a) in a single vessel, providing a reaction mixture comprising the nucleic acid sample of the subject, a polymerizing enzyme and a probe set, which probe set comprises (i) a first probe that has sequence specificity for an SMN1 gene at a first locus of the nucleic acid sample, (ii) a second probe that has sequence specificity for an SMN2 gene at the first locus, (iii) a third probe that has sequence specificity for the SMN1 or SMN2 gene at a second locus of the nucleic acid sample, which second locus is different than the first locus, and (iv) a fourth probe that has sequence specificity for a genetic aberration of the SMN1 gene at the second locus; (b) subjecting the reaction mixture in the single vessel to conditions sufficient to generate a plurality of amplicons corresponding to the first locus and the second locus; (c) detecting the plurality of amplicons; and (d) based at least in part on the plurality of amplicons detected in (c), (i) identifying the genetic signature(s) associated with SMA at an accuracy of at least 90%.

In some embodiments, the method comprises in (d) identifying (i) a copy number in SMN1 or (ii) the genetic aberration of the SMN1 gene. In some embodiments, the method comprises in (d) identifying (i) a copy number in SMN1 and (ii) the genetic aberration of the SMN1 gene.

In some embodiments, the method comprises in (c) measuring a plurality of intensities corresponding to the first probe, second probe, third probe and fourth probe. In some embodiments, the method further comprises measuring the plurality of intensities against an intensity from a control probe.

In some embodiments, the method comprises in (b) performing a polymerase chain reaction on the nucleic acid sample at the first locus and the second locus. In some embodiments, the reaction mixture comprises primers targeting the first locus and the second locus.

In some embodiments, the detecting comprises detecting optical signals corresponding to the plurality of amplicons. In some embodiments, the optical signals are fluorescent signals.

In another aspect, the disclosure provides a system for identifying a genetic signature(s) associated with spinal muscular atrophy (SMA) in a nucleic acid sample of a subject, comprising: (a) a single vessel configured to contain a reaction mixture comprising the nucleic acid sample of the subject, a polymerizing enzyme and a probe set, which probe set comprises (i) a first probe that has sequence specificity for an SMN1 gene at a first locus of the nucleic acid sample, (ii) a second probe that has sequence specificity for an SMN2 gene at the first locus, (iii) a third probe that has sequence specificity for the SMN1 or SMN2 gene at a second locus of the nucleic acid sample, which second locus is different than the first locus, and (iv) a fourth probe that has sequence specificity for a genetic aberration of the SMN1 gene at the second locus; (b) a detector operatively coupled to the single vessel; and (c) one or more computer processors operatively coupled to the single vessel, and the one or more computer processors are individually or collectively programmed to (i) subject the reaction mixture in the single vessel to conditions sufficient to generate a plurality of amplicons corresponding to the first locus and the second locus; (ii) use the detector to detect the plurality of amplicons; and (iii) based at least in part on the plurality of amplicons detected in (ii), identify the genetic signature(s) associated with SMA at an accuracy of at least 90%.

In some embodiments, the one or more computer processors are individually or collectively programmed to identify (i) a copy number in SMN1 or (ii) the genetic aberration of the SMN1 gene. In some embodiments, the one or more computer processors are individually or collectively programmed to identify (i) a copy number in SMN1 and (ii) the genetic aberration of the SMN1 gene.

In some embodiments, the detector is an optical detector.

In some embodiments, the system further comprises a heating unit in thermal communication with the single vessel. In some embodiments, the one or more computer processors are individually or collectively programmed to direct the heating unit to subject the reaction mixture to one or more heating and cooling cycles to generate the plurality of amplicons.

In some embodiments, the system further comprises a heating unit in thermal communication with the single vessel, and the one or more computer processors are individually or collectively programmed to direct the heating unit to subject the reaction mixture to heating to generate the plurality of amplicons.

In some embodiments, the heating is isothermal heating.

In some embodiments, the nucleic acid sample is obtained from the subject and provided in the single vessel without any filtration, extraction or purification.

In some embodiments, the nucleic acid sample is a chromosome or a derivative of the chromosome.

In yet another aspect, the disclosure provides a kit for identifying a genetic signature(s) associated with spinal muscular atrophy (SMA) in a nucleic acid sample of a subject, comprising a probe set comprising: (a) a first probe that has sequence specificity for an SMN1 gene at a first locus of a nucleic acid sample of the subject; (b) a second probe that has sequence specificity for an SMN2 gene at the first locus; (c) a third probe that has sequence specificity for the SMN1 or SMN2 gene at a second locus of the nucleic acid sample, which second locus is different than the first locus; and (d) a fourth probe that has sequence specificity for a genetic aberration of the SMN1 gene at the second locus; and (e) instructions for using said probe set to identify the genetic signature associated with SMA, at an accuracy of at least 90%.

In some embodiments, the instructions direct a user to (i) provide, in a single vessel, a reaction mixture comprising a nucleic acid sample of the subject, a polymerizing enzyme and the probe set, (ii) subject the reaction mixture in the single vessel to conditions sufficient to generate a plurality of amplicons corresponding to the first locus and the second locus, (iii) detect the plurality of amplicons, and (iv) based at least in part on the plurality of amplicons detected in (c), identify the SMA in the subject at an accuracy of at least 90%. In some embodiments, the instructions direct the user to identify (i) a copy number in SMN1 or (ii) the genetic aberration of the SMN1 gene, to identify the genetic signature(s) associated with SMA at the accuracy of at least 90%. In some embodiments, the instructions direct the user to identify (i) a copy number in SMN1 and (ii) the genetic aberration of the SMN1 gene, to identify the genetic signature(s) associated with SMA with an accuracy of at least 90%.

In some embodiments, the genetic aberration of the SMN1 gene is a two-copy haplotype.

In some embodiments, the probe set further comprises a fifth probe that is configured to provide a control signal. In some embodiments, the control signal is an optical signal.

In some embodiments the first probe, second probe, third probe and fourth probe are quantitative polymerase chain (qPCR) reaction probes. In some embodiments, the first probe, second probe, third probe and fourth probe are hydrolysis probes. In some embodiments, the hydrolysis probes are TaqMan™ probes. In some embodiments, the first probe, second probe, third probe and fourth probe are molecular beacons. In some embodiments, the first probe, second probe, third probe and fourth probe are primers for performing nucleic acid amplification reactions at the first locus and the second locus.

In some embodiments, the first probe, second probe, third probe and fourth probe are configured to emit different signals. In some embodiments, the different signals are different optical signals.

In some embodiments, at least one of the first probe, second probe, third probe and fourth probe comprises at least one locked nucleic acid (LNA). In some embodiments, at least one of the first probe, second probe, third probe and fourth probe comprises at least two locked nucleic acids. In some embodiments, at least one of the first probe, second probe, third probe and fourth probe comprises at least three locked nucleic acids. In some embodiments, at least one of the first probe, second probe, third probe and fourth probe comprises at least four locked nucleic acids. In some embodiments, at least one of the first probe, second probe, third probe and fourth probe comprises at least five locked nucleic acids. In some embodiments, at least one of the first probe, second probe, third probe and fourth probe comprises at least six locked nucleic acids.

In some embodiments, each of at least two of the first probe, second probe, third probe and fourth probe comprises at least one locked nucleic acid. In some embodiments, each of at least three of the first probe, second probe, third probe and fourth probe comprises at least one locked nucleic acid. In some embodiments each of the first probe, second probe, third probe and fourth probe comprises at least one locked nucleic acid.

In some embodiments, the accuracy is at least 95%. In some embodiments, the accuracy is at least 98%.

Another aspect of the present disclosure provides a non-transitory computer-readable medium comprising machine-executable code that, upon execution by one or more computer processors, implements any of the methods above or elsewhere herein.

Another aspect of the present disclosure provides a system comprising one or more computer processors and computer memory coupled thereto. The computer memory comprises machine-executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “Fig.” herein), of which:

FIG. 1 is schematic depicting an example system of the present disclosure.

FIG. 2 depicts a schematic of an example assay for detecting copy number of SMN1 and SMN2 and detecting the two-copy haplotype.

FIGS. 3A, 3B, and 3C are graphs depicting results of monitoring example nucleic acid amplification reactions described in Example 1 where a set of nucleic acid samples are used comprising a copy number of SMN1 genes and the two-copy haplotype.

FIG. 4 is a graph depicting results of example nucleic acid amplification reactions described in Example 1 where the nucleic acid sample comprises no SMN1 genes

FIG. 5 depicts a two-axis chart of the general signal of samples comprising or lacking the two-copy haplotype.

FIG. 6 is a graph depicting results of monitoring example nucleic acid amplification reactions to detect the two-copy haplotype.

FIG. 7 is a schematic of an example electronic display having an example user interface.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an”, and “the” include plural references unless the context clearly dictates otherwise. For example, the term “a cell” includes a plurality of cells, including mixtures thereof.

As used herein, the terms “amplifying” and “amplification” are used interchangeably and generally refer to generating one or more copies of “amplified product” or “amplicon” of a nucleic acid. The terms “amplified product” and “amplicon” may be used interchangeably. The term “DNA amplification” generally refers to generating one or more copies of a DNA molecule or “amplified DNA product”.

As used herein, the term “cycle threshold” or “Ct” generally refers to the cycle during thermocycling in which an increase in a detectable signal due to amplified product reaches a statistically significant level above background signal.

As used herein, the terms “denaturing” and “denaturation” are used interchangeably and generally refer to the full or partial unwinding of the helical structure of a double-stranded nucleic acid, and in some cases the unwinding of the secondary structure of a single-stranded nucleic acid. Denaturation may include the inactivation of the cell wall(s) of a pathogen or the shell of a virus, and the inactivation of the protein(s) of inhibitors. Conditions at which denaturation may occur include a “denaturation temperature” that generally refers to a temperature at which denaturation may occur and a “denaturation duration” that generally refers to an amount of time allotted for denaturation to occur.

As used herein, the term “elongation” generally refers to the incorporation of nucleotides to a nucleic acid in a template directed fashion. Elongation may occur via the aid of an enzyme, such as, for example, a polymerase or reverse transcriptase. Conditions at which elongation may occur include an “elongation temperature” that generally refers to a temperature at which elongation may occur and an “elongation duration” that generally refers to an amount of time allotted for elongation to occur.

As used herein, the term “nucleic acid” generally refers to a polymeric form of nucleotides of any length, either deoxyribonucleotides (dNTPs) or ribonucleotides (rNTPs), or analogs thereof. Nucleic acids may have any three dimensional structure, and may perform any function, known or unknown. Non-limiting examples of nucleic acids include DNA, RNA, coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent.

As used herein, the term “primer extension reaction” generally refers to the denaturing of a double-stranded nucleic acid, binding of a primer to one or both strands of the denatured nucleic acid, followed by elongation of the primer(s).

As used herein, the term “reaction mixture” generally refers to a composition comprising reagents used to complete nucleic acid amplification (e.g., DNA amplification, RNA amplification), with non-limiting examples of such reagents that include primer sets having specificity for target RNA or target DNA, DNA produced from reverse transcription of RNA, a DNA polymerase, a reverse transcriptase (e.g., for reverse transcription of RNA), suitable buffers (including zwitterionic buffers), co-factors (e.g., divalent and monovalent cations), dNTPs, and other enzymes (e.g., uracil-DNA glycosylase (UNG)), etc). In some cases, reaction mixtures can also comprise one or more reporter agents.

As used herein, a “reporter agent” generally refers to a composition that yields a detectable signal, the presence or absence of which can be used to detect the presence of amplified product.

As used herein, the term “target nucleic acid” generally refers to a nucleic acid molecule in a starting population of nucleic acid molecules having a nucleotide sequence whose presence, amount, and/or sequence, or changes in one or more of these, are desired to be determined. A target nucleic acid may be any type of nucleic acid, including DNA, RNA, and analogues thereof. As used herein, a “target ribonucleic acid (RNA)” generally refers to a target nucleic acid that is RNA. As used herein, a “target deoxyribonucleic acid (DNA)” generally refers to a target nucleic acid that is DNA.

As used herein, the term “subject,” generally refers to an entity or a medium that has testable or detectable genetic information. A subject can be a person or individual. A subject can be a vertebrate, such as, for example, a mammal. Non-limiting examples of mammals include murines, simians, humans, farm animals, sport animals, and pets. Other examples of subjects include food, plant, soil, and water.

As used herein, the term “locked nucleic acid” or “LNA,” generally refers to a nucleic acid comprising a nucleotide which provides a greater thermodynamic stability upon hybridization as compared to a thermodynamic stability of hybridization of a nucleic acid in which an unmodified nucleotide is in place of the modified nucleotide. A locked nucleic acid may contain additional bonds and atoms that “lock” the nucleic acid into a conformation that is favorable for hybridization. The additional bonds and atoms may be, for example, additional bonds and atoms bridging the 2′ oxygen and the 4′ carbon of the ribose.

As used herein, the term “probe” refers to a nucleic acid molecule that allows the detection of sequences via hybridization or a binding interaction. The probe may include a reporter agent which allows the hybridization or binding interaction to be detected. The probe may be at least 2, 3, 4, 5, 6, 7, 8, 9, 10, 12, 14, 16, 18, 20, 30, 40, 50, 60, 70, 80, 90, 100, or more nucleotides long. The probe may be at most 100, 90, 80, 70, 60, 50, 40, 30, 20, 18, 16, 14, 12, 10, 9, 8, 7, 6, 5, 4, 3, 2 or less nucleotides long

As used herein, the term “genetic aberration” generally refers to a difference in the DNA sequence of a gene or chromosome of an individual compared to that of a reference healthy individual or a wild type sample. A genetic aberration, may be, for example a point mutation, an insertion, a deletion, a transposition, a gene duplication, or other changes of a nucleic acid sequence of a chromosome. A genetic aberration, for example, may cause a change in the expression level of a gene, a change in the sequence of a polypeptide encoded by a gene, a change in function of a polypeptide encoded by a gene, or a loss of function of a polypeptide encoded by a gene. Multiple genetic aberrations may occur such that the effect of one genetic aberration is counteracted by another genetic aberration. For example, a gene deletion may occur and is accompanied by a gene mutation in a different copy or similar gene such that the overall gene expression is unchanged. A genetic aberration, for example, may still be present in the chromosome of a seemingly healthy individual, with the genetic aberration becoming more apparent in the form of a disease or disorder when the genetic material is passed on to the offspring.

As used herein, the term “haplotype” generally refers to a group of alleles that are inherited together from a parent or passed on together from a parent. This group of alleles, for example, is close to one another of the chromosome and so is passed on together.

As used herein, the term “two-copy haplotype” generally refers to a haplotype that comprises at least two copies of the SMN1 gene found on the same copy of a chromosome. This two-copy haplotype may be, for example, the result of gene duplication event, a gene insertion, or a gene mutation, that results in two copies of SMN1 gene on the same chromosome. In some cases, a two-copy haplotype may be a multi-copy haplotype (e.g., three or more copies).

As used herein, the term “carrier” generally refers to an individual that has an allele or genetic aberration that is causative or correlated with a disease or disorder. The individual may appear to be healthy and not display traits or symptoms of the disease. The individual can pass the allele to the individual's offspring in which the offspring may display traits or symptoms of the disease or disorder. A carrier of the two-copy haplotype, for example, may appear to be healthy and not display symptoms of SMA. The carrier's offspring may display symptoms of SMA, due to the passing of an allele correlated with SMA.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Spinal muscular atrophy (SMA) in a subject (e.g., human) may be caused by a genetic deficiency related to the SMN1 gene. The genetic deficiency may result in a deficiency of the SMN1 protein. Gene mutations in the SMN1 gene can result in a non-functional SMN1 gene which may in turn result in production of non-functional SMN1 protein. Additionally, subjects that have SMA may be missing the SMN1 gene altogether. Detecting gene variants and copy number of SMN1 can help identify SMA in a subject or determine if the subject is a carrier of SMA. A seemingly healthy subject may also be a carrier for SMA through a “silent” or (2-0) allele. A subject may have a sufficient number of SMN1 genes, for example, one on each chromosome, and pass on one copy to their progeny. However, the subject may also have two copies on one chromosome and zero copies on the other chromosome. This subject, when passing on their genes to their offspring, may pass on the chromosome segment that contains zero copies, thus giving rise to SMA in their offspring when the offspring inherits a deficient chromosome from the second parent.

Additionally, when detecting the presence of the SMN1 gene, it may be difficult to differentiate the SMN1 gene from the SMN2 gene. The SMN2 gene is closely related to and has a high sequence homology with the SMN1 gene. For example, the wild type SMN1 and SMN2 genes may differ by only a few nucleotides. Because of the similarity between SMN1 and SMN2, it is difficult to accurately determine copy number of SMN1 versus SMN2. The detection of haplotypes representing the two-copy haplotype may have similar detection difficulties due to the sequence similarity of those with or without the two-copy haplotype. As such, there is interest in accurately identifying carriers of the two-copy haplotype and accurately determining copy number for both SMN1 and SMN2.

In an aspect, the disclosure provides a kit for identifying a genetic signature associated with spinal muscular atrophy (SMA) in a subject or identifying the subject as a carrier of SMA. The kit may comprise a probe set. The probe set may comprise a first probe that has sequence specificity for an SMN1 gene at a first locus of a nucleic acid sample of the subject; a second probe that has sequence specificity for an SMN2 gene at the first locus; a third probe that has sequence specificity for the SMN1 or SMN2 gene at a second locus of the nucleic acid sample, which second locus is different than the first locus; and a fourth probe that has sequence specificity for a genetic aberration of the SMN1 gene at the second locus. The kit may comprise instructions for using the probe set to identify the SMA in the subject at an accuracy of at least 90%. The probe set may be in a lyophilized format. The kit may comprise the probe set and additional reagents including salts, buffers, sugars, enzymes, primers, nucleotides, or a combination thereof. The kit may comprise a probe set and additional reagents in which the probe set and additional reagents are provided in a single vessel. The probe set and additional reagents may be lyophilized together and provided in a single vessel. The kit may comprise a diluent for rehydrating the lyophilized components.

In some embodiments, the instructions direct a user to (i) provide, in a single vessel, a reaction mixture comprising a nucleic acid sample of the subject, a polymerizing enzyme and the probe set, (ii) subject the reaction mixture in the single vessel to conditions sufficient to generate a plurality of amplicons corresponding to the first locus and the second locus, (iii) detect the plurality of amplicons, and (iv) based at least in part on the plurality of amplicons detected in (c), identify the SMA in the subject with an accuracy of at least 70%, 80%, 91%, 92%, 93%, 94%, 95%, 96%, 97%, 98%, 99%, 99.9%. In some embodiments, the instructions direct the user to identify (i) a copy number in SMN1 or (ii) the genetic aberration of the SMN1 gene, to identify the SMA in the subject with an accuracy of at least 70%, 80%, 91%, 92%, 93%, 94%, 95%, 96%, 97%, 98%, 99%, 99.9%. In some embodiments, the instructions direct the user to identify (i) a copy number in SMN1 and (ii) the genetic aberration of the SMN1 gene, identify the SMA in the subject with an accuracy of at least 70%, 80%, 91%, 92%, 93%, 94%, 95%, 96%, 97%, 98%, 99%, 99.9%.

In an aspect, the disclosure provides a method for identifying genetic signature associated with spinal muscular atrophy (SMA) in a subject or identifying the subject as a carrier of SMA. The method comprises providing a reaction mixture in a single vessel comprising a nucleic acid sample of the subject, a polymerizing enzyme and a probe set. The reaction mixture may be in a lyophilized starting format. The probe set may comprise a first probe that has sequence specificity for an SMN1 gene at a first locus of the nucleic acid sample, a second probe that has sequence specificity for an SMN2 gene at the first locus, a third probe that has sequence specificity for the SMN1 or SMN2 gene at a second locus of the nucleic acid sample, which second locus is different than the first locus, and a fourth probe that has sequence specificity for a genetic aberration of the SMN1 gene at the second locus. The method may comprise subjecting the reaction mixture in the single vessel to conditions sufficient to generate a plurality of amplicons corresponding to the first locus and the second locus and detecting the plurality of amplicons. The method may comprise identifying the SMA in the subject at an accuracy of at least 90%, based at least in part on the plurality of amplicons detected

The method may comprise performing a polymerase chain reaction on the nucleic acid sample at the first locus and the second locus. The reaction mixture may comprise primers targeting the first locus and the second locus. The method may further comprise performing a polymerase chain reaction at a third locus. The control probe may bind to the amplicon generated by performing the polymerase reaction at the third locus. This binding of the control probe to the third locus, may contribute to increasing the accuracy of identifying the copy number in SMN1 or SMN2.

The method may comprise measuring a plurality of intensities corresponding to the first probe, second probe, third probe and fourth probe. The method may include measuring the plurality of intensities against an intensity from a control probe. The method may include measuring greater than or equal to 1, 2, 3, 4, or 5 intensities. The method may include measuring greater at least 5, 10, 15, 20, 30, 40, 50, or more intensities over multiple samples or vessels. The method may include measuring the intensities individually, sequentially, or both sequentially and simultaneously. In an example, at least two intensities of the plurality of intensities are measured simultaneously. In an example, at least three intensities of the plurality of intensities are measured simultaneously. In an example, at least four intensities of the plurality of intensities are measured simultaneously. In an example, five intensities of the plurality of intensities are measured simultaneously. In some examples, the intensities each correspond to a different wavelength of light. In some examples, the intensities may correspond to the same wavelength. The intensities may correspond to a wavelength or a wavelength range. In some cases, portions of the intensities may correspond to one wavelength or wavelength range and another portion of the intensities corresponds to another wavelength or wavelength range.

The method may comprise identifying a copy number in SMN1 or the genetic aberration of the SMN1 gene. The method may comprise identifying a copy number in SMN1 and the genetic aberration of the SMN1 gene. The method may comprise identifying a copy number in SMN2. In some cases, identifying the copy number in SMN1 is sufficient to identify a subject as having SMA. In other cases, identifying the genetic aberration of the SMN1 gene is sufficient to identify a subject as having SMA. In other cases, identifying the genetic aberration of the SMN1 gene is sufficient to identify a subject as a carrier of SMA

In various aspects, the time required to complete the elements of a method may vary depending upon the particular steps of the method. For example, an amount of time for completing the elements of a method may be from about 5 minutes to about 120 minutes. In other examples, an amount of time for completing the elements of a method may be from about 5 minutes to about 60 minutes. In other examples, an amount of time for completing the elements of a method may be from about 5 minutes to about 30 minutes. In other examples, an amount of time for completing the elements of a method may be less than or equal to 120 minutes, less than or equal to 90 minutes, less than or equal to 75 minutes, less than or equal to 60 minutes, less than or equal to 45 minutes, less than or equal to 40 minutes, less than or equal to 35 minutes, less than or equal to 30 minutes, less than or equal to 25 minutes, less than or equal to 20 minutes, less than or equal to 15 minutes, less than or equal to 10 minutes, or less than or equal to 5 minutes.

In another aspect, the disclosure provides a system for identifying a genetic signature associated with spinal muscular atrophy (SMA) in a subject or identifying the subject as a carrier of SMA. The system may comprise a single vessel configured to contain a reaction mixture comprising a nucleic acid sample of the subject, a polymerizing enzyme and a probe set (e.g., in a lyophilized format). The probe set may comprises a first probe that has sequence specificity for an SMN1 gene at a first locus of the nucleic acid sample, a second probe that has sequence specificity for an SMN2 gene at the first locus, a third probe that has sequence specificity for the SMN1 or SMN2 gene at a second locus of the nucleic acid sample, which second locus is different than the first locus, and a fourth probe that has sequence specificity for a genetic aberration of the SMN1 gene at the second locus. The system may comprise a detector operatively coupled to the single vessel. The system may comprise one or more computer processors operatively coupled to the single vessel, in which the one or more computer processors are individually or collectively programmed to subject the reaction mixture in the single vessel to conditions sufficient to generate a plurality of amplicons corresponding to the first locus and the second locus; The system may comprise using the detector to detect the plurality of amplicons, and based at least in part on the plurality of amplicons detected, identify the SMA in the subject at an accuracy of at least 90%.

In some embodiments, the detector is an optical detector. Optical detection methods include, but are not limited to, fluorimetry and UV-vis light absorbance. Spectroscopic detection methods include, but are not limited to, mass spectrometry, nuclear magnetic resonance (NMR) spectroscopy, and infrared spectroscopy. Electrostatic detection methods include, but are not limited to, gel based techniques, such as, for example, gel electrophoresis. Electrochemical detection methods include, but are not limited to, electrochemical detection of amplified product after high-performance liquid chromatography separation of the amplified products. For example, the optical detector is used to detect the emission of light. The light may be of different wavelengths and the optical detector is set to detect a range of wavelengths or a specific wavelength. In some cases, the optical detector can detect intensity for each wavelength range. Each probe can create an optical signal such that the optical detector can detect it. Each probe can additionally create an optical signal that emits a different wavelength from one another. The optical detector may detect signals simultaneously. Alternatively, or in addition to, the optical detector may detect signals simultaneously. The optical detector may detect signals at different wavelengths. The optical detector may detect signals a specific wavelength or wavelength range.

The system may further comprise a heating unit in thermal communication with the single vessel, in which the one or more computer processors are individually or collectively programmed to direct the heating unit to subject the reaction mixture to one or more heating and cooling cycles to generate the plurality of amplicons, The system further comprises a heating unit in thermal communication with the single vessel, in which the one or more computer processors are individually or collectively programmed to direct the heating unit to subject the reaction mixture to heating to generate the plurality of amplicons. The heating unit may perform isothermal heating.

The heating unit may be used to denature the nucleic acids. Denaturation temperatures may vary depending upon, for example, the particular nucleic acid sample analyzed, the particular source of target nucleic acid (e.g., in tissue, cell free, plasma) of the nucleic acid sample, the reagents used, and/or the desired reaction conditions. For example, a denaturation temperature may be from about 80° C. to about 110° C. In some examples, a denaturation temperature may be from about 90° C. to about 100° C. In some examples, a denaturation temperature may be from about 90° C. to about 97° C. In some examples, a denaturation temperature may be from about 92° C. to about 95° C. In still other examples, a denaturation temperature may be about 80°, 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

Denaturation durations may vary depending upon, for example, the particular nucleic acid sample analyzed, the particular source of target nucleic acid (e.g., in tissue, cell free, plasma) of the nucleic acid sample, the reagents used, and/or the desired reaction conditions. For example, a denaturation duration may be less than or equal to 300 seconds, 240 seconds, 180 seconds, 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. For example, a denaturation duration may be no more than 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second.

The heating unit may be used to create temperatures suitable for elongation of nucleic acids by a polymerase. Elongation temperatures may vary depending upon, for example, the particular nucleic acid sample analyzed, the particular source of target nucleic acid (e.g., in tissue, cell free, plasma) of the nucleic acid sample, the reagents used, and/or the desired reaction conditions. For example, an elongation temperature may be from about 30° C. to about 80° C. In some examples, an elongation temperature may be from about 35° C. to about 72° C. In some examples, an elongation temperature may be from about 45° C. to about 65° C. In some examples, an elongation temperature may be from about 35° C. to about 65° C. In some examples, an elongation temperature may be from about 40° C. to about 60° C. In some examples, an elongation temperature may be from about 50° C. to about 60° C. In still other examples, an elongation temperature may be about 35°, 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., or 80° C.

Elongation durations may vary depending upon for example, the particular nucleic acid sample analyzed, the particular source of target nucleic acid (e.g., in tissue, cell free, plasma) of the nucleic acid sample, the reagents used, and/or the desired reaction conditions. For example, an elongation duration may be less than or equal to 300 seconds, 240 seconds, 180 seconds, 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. For example, an elongation duration may be no more than 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second.

In some embodiments, the ramping time (i.e., the time the heating unit takes to transition from one temperature to another) and/or ramping rate can be important factors in amplification. For example, the temperature and time for which amplification yields a detectable amount of amplified product indicative of the presence of a target nucleic acid can vary depending upon the ramping rate and/or ramping time. The ramping rate can impact the temperature(s) and time(s) used for amplification.

In some cases, the ramping time and/or ramping rate can be different between cycles. In some situations, however, the ramping time and/or ramping rate between cycles can be the same. The ramping time and/or ramping rate can be adjusted based on the sample(s) that are being processed.

In some situations, the ramping time between different temperatures can be determined, for example, based on the nature of the sample and the reaction conditions. The exact temperature and incubation time can also be determined based on the nature of the sample and the reaction conditions. In some embodiments, a single sample can be processed (e.g., subjected to amplification conditions) multiple times using multiple thermal cycles, with each thermal cycle differing for example by the ramping time, temperature, and/or incubation time. The best or optimum thermal cycle can then be chosen for that particular sample. This provides a robust and efficient method of tailoring the thermal cycles to the specific sample or combination of samples being tested.

In some embodiments, a target nucleic acid may be subjected to a denaturing condition prior to initiation of a primer extension reaction. In the case of a plurality of series of primer extension reactions, the target nucleic acid may be subjected to a denaturing condition prior to executing the plurality of series or may be subjected to a denaturing condition between series of the plurality. For example, the target nucleic acid may be subjected to a denaturing condition between a first series and a second series of a plurality of series. Non-limiting examples of such denaturing conditions include a denaturing temperature profile (e.g., one or more denaturing temperatures) and a denaturing agent.

In some embodiments, a nucleic acid sample may be preheated prior to conducting a primer extension reaction. The temperature (e.g., a preheating temperature) at which and duration (e.g., a preheating duration) for which a nucleic acid sample is preheated may vary depending upon, for example, the particular nucleic acid being analyzed. In some examples, a nucleic acid sample may be preheated for no more than about 60 minutes, 50 minutes, 40 minutes, 30 minutes, 25 minutes, 20 minutes, 15 minutes, 10 minutes, 9 minutes, 8 minutes, 7 minutes, 6 minutes, 5 minutes, 4 minutes, 3 minutes, 2 minutes, 1 minute, 45 seconds, 30 seconds, 20 seconds, 15 seconds, 10 seconds, or 5 seconds. In some examples, a nucleic acid sample may be preheated at a temperature from about 80° C. to about 110° C. In some examples, a nucleic acid sample may be preheated at a temperature from about 90° C. to about 100° C. In some examples, a nucleic acid sample may be preheated at a temperature from about 90° C. to about 97° C. In some examples, a nucleic acid sample may be preheated at a temperature from about 92° C. to about 95° C. In still other examples, a nucleic acid may be preheated at a temperature of about 80°, 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

In some embodiments, the one or more computer processors are individually or collectively programmed to identify (i) a copy number in SMN1 or (ii) the genetic aberration of the SMN1 gene. In some embodiments, the one or more computer processors are individually or collectively programmed to identify (i) a copy number in SMN1 and (ii) the genetic aberration of the SMN1 gene.

In various aspects, methods and systems described herein are useful for a genetic signature(s) associated with SMA with high accuracy. The accuracy of identifying the genetic signature(s) associated with SMA may be at least 90%. The accuracy of identifying the genetic signature(s) associated with may be at least 95%. The accuracy of identifying the genetic signature(s) associated with SMA may be at least 70%, 80%, 90%, 91%, 92%, 93%, 94%, 95%, 96%, 97%, 98%, 99%, 99.9%, or higher.

Kits, methods and systems of the present disclosure may be used to identify genetic signature(s) associated with SMA at a sensitivity of at least 70%, 80%, 90%, 91%, 92%, 93%, 94%, 95%, 96%, 97%, 98%, 99%, 99.9%, or higher. Kits, methods and systems of the present disclosure may be used to identify genetic signature(s) associated with SMA at a specificity of at least 70%, 80%, 90%, 91%, 92%, 93%, 94%, 95%, 96%, 97%, 98%, 99%, 99.9%, or higher.

In any of the various aspects, a genetic signature associated with SMA is identified in a subject. The genetic signature may be one or more genetic signature. In some examples, the genetic signature is (i) a copy number of an SMN1 gene and/or an SMN2 gene, and/or (ii) a genetic aberration in the SMN1 gene and/or SMN2 gene. In some cases, the genetic signature may be for an individual having or suspected of having SMA. The genetic signature may indicate that the individual has SMA. In some cases, the genetic signature may be for an unaffected individual (i.e., an individual who does not display SMA) who may have an increased risk of having offspring affected with SMA. The genetic signature may indicate that the individual has an increased risk of having offspring affected with SMA.

In various aspects, a copy number is identified. In an example, a copy number of 0, 1, 2, or more may be identified for SMN1 and/or SMN2. In another example, a variation in copy number (e.g., a copy number increase or decrease) may be identified.

In any of the various aspects, primer sets directed to a target nucleic acid may be utilized to conduct nucleic acid amplification reaction. In such cases, the primer set may be a primer set specifically designed to amplify one or more sequences of the target nucleic acid molecule. In some embodiments, the amplification protocol may further include selecting a reporter agent (e.g., an oligonucleotide probe comprising an optically-active species or other type of reporter agent described elsewhere herein) that is specific for one or more sequences of the target nucleic acid molecule. Moreover, in some embodiments, the reagents may comprise any suitable reagents used for nucleic acid amplification as described elsewhere herein, such as, for example, a deoxyribonucleic acid (DNA) polymerase, a primer set for the target nucleic acid, and (optionally) a reverse transcriptase.

Primer sets generally comprise one or more primers. For example, a primer set may comprise about 1, 2, 3, 4, 5, 6, 7, 8, 9, 10, or more primers. In some cases, a primer set or may comprise primers directed to different amplified products or different nucleic acid amplification reactions. For example, a primer set may comprise a first primer used to generate a first strand of nucleic acid product that is complementary to at least a portion of the target nucleic acid and a second primer complementary to the nucleic acid strand product used to generate a second strand of nucleic acid product that is complementary to at least a portion of the first strand of nucleic acid product.

Where desired, any suitable number of primer sets may be used. For example, about 1, 2, 3, 4, 5, 6, 7, 8, 9, 10, or more primer sets may be used. Where multiple primer sets are used, one or more primer sets may each correspond to a particular nucleic acid amplification reaction or amplified product.

In various aspects, primer extension reactions are utilized to generate amplified product. Primer extension reactions generally comprise a cycle of incubating a reaction mixture at a denaturation temperature for a denaturation duration and incubating a reaction mixture at an elongation temperature for an elongation duration. In any of the various aspects, multiple cycles of a primer extension reaction can be conducted. Any suitable number of cycles may be conducted. For example, the number of cycles conducted may be less than about 100, 90, 80, 70, 60, 50, 40, 30, 20, 10, or 5 cycles. The number of cycles conducted may depend upon, for example, the number of cycles (e.g., cycle threshold (Ct) value) used to obtain a detectable amplified product (e.g., a detectable amount of amplified DNA product that is indicative of the presence of a target DNA in a nucleic acid sample). For example, the number of cycles used to obtain a detectable amplified product (e.g., a detectable amount of DNA product that is indicative of the presence of a target DNA in a nucleic acid sample) may be less than about or about 100 cycles, 75 cycles, 70 cycles, 65 cycles, 60 cycles, 55 cycles, 50 cycles, 40 cycles, 35 cycles, 30 cycles, 25 cycles, 20 cycles, 15 cycles, 10 cycles, or 5 cycles. Moreover, in some embodiments, a detectable amount of an amplifiable product (e.g., a detectable amount of DNA product that is indicative of the presence of a target DNA in a nucleic acid sample) may be obtained at a cycle threshold value (Ct) of less than 100, 75, 70, 65, 60, 55, 50, 45, 40, 35, 30, 25, 20, 15, 10, or 5.

The time for which amplification yields a detectable amount of amplified product can vary depending upon the nucleic sample, the particular nucleic acid amplification reactions to be conducted, and the particular number of cycles of amplification reaction desired. For example, amplification of a target nucleic acid may yield a detectable amount of amplified product indicative to the presence of the target nucleic acid at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

In some embodiments, amplification of a nucleic acid may yield a detectable amount of amplified DNA at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

In some embodiments, a reaction mixture may be subjected to a plurality of series of primer extension reactions. An individual series of the plurality may comprise multiple cycles of a particular primer extension reaction, characterized, for example, by particular denaturation and elongation conditions as described elsewhere herein. Generally, each individual series differs from at least one other individual series in the plurality with respect to, for example, a denaturation condition and/or elongation condition. An individual series may differ from another individual series in a plurality of series, for example, with respect to any one, two, three, or all four of denaturing temperature, denaturing duration, elongation temperature, and elongation duration. Moreover, a plurality of series may comprise any number of individual series such as, for example, at least about or about 2, 3, 4, 5, 6, 7, 8, 9, 10, or more individual series.

For example, a plurality of series of primer extension reactions may comprise a first series and a second series. The first series, for example, may comprise more than ten cycles of a primer extension reaction, where each cycle of the first series comprises (i) incubating a reaction mixture at about 92° C. to about 95° C. for no more than 30 seconds followed by (ii) incubating the reaction mixture at about 35° C. to about 65° C. for no more than about one minute. The second series, for example, may comprise more than ten cycles of a primer extension reaction, where each cycle of the second series comprises (i) incubating the reaction mixture at about 92° C. to about 95° C. for no more than 30 seconds followed by (ii) incubating the reaction mixture at about 40° C. to about 60° C. for no more than about 1 minute. In this particular example, the first and second series differ in their elongation temperature condition. The example, however, is not meant to be limiting as any combination of different elongation and denaturing conditions may be used.

An advantage of conducting a plurality of series of primer extension reaction may be that, when compared to a single series of primer extension reactions under comparable denaturing and elongation conditions, the plurality of series approach yields a detectable amount of amplified product that is indicative of the presence of a target nucleic acid in a biological sample with a lower cycle threshold value. Use of a plurality of series of primer extension reactions may reduce such cycle threshold values by at least about or about 1%, 5%, 10%, 15%, 20%, 25%, 30%, 35%, 40%, 45%, 50%, 55%, 60%, 65%, 70%, 75%, 80%, 85%, 90%, or 95% when compared to a single series under comparable denaturing and elongation conditions.

In any of the various aspects, a DNA polymerase is used. Any suitable DNA polymerase may be used, including commercially available DNA polymerases. A DNA polymerase generally refers to an enzyme that is capable of incorporating nucleotides to a strand of DNA in a template bound fashion. Non-limiting examples of DNA polymerases include Taq polymerase, Tth polymerase, Tli polymerase, Pfu polymerase, VENT® polymerase, DEEPVENT® polymerase, EX-Taq polymerase, LA-Taq™ polymerase, Expand™ polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, PfuTurbo polymerase, Pyrobest™ polymerase, Pwo polymerase, KOD polymerase, Bst polymerase, Sac polymerase, Klenow fragment, and variants, modified products and derivatives thereof. For certain Hot Start Polymerase, a denaturation step at 94° C. to 95° C. for 2 minutes to 10 minutes may be required, which may change the thermal profile based on different polymerases.

Any type of nucleic acid amplification reaction may be used to amplify a target nucleic acid and generate an amplified product. Moreover, amplification of a nucleic acid may linear, exponential, or a combination thereof. Amplification may be emulsion based or may be non-emulsion based. Non-limiting examples of nucleic acid amplification methods include reverse transcription, primer extension, polymerase chain reaction, ligase chain reaction, helicase-dependent amplification, asymmetric amplification, rolling circle amplification, and multiple displacement amplification (MDA). In some embodiments, the amplified product may be DNA. In cases where DNA is amplified, any DNA amplification method may be employed. Non-limiting examples of DNA amplification methods include polymerase chain reaction (PCR), variants of PCR (e.g., real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, touchdown PCR), and ligase chain reaction (LCR). In some cases, DNA amplification is linear. In some cases, DNA amplification is exponential.

In various aspects, nucleic acid amplification reactions described herein may be conducted in parallel. In general, parallel amplification reactions are amplification reactions that occur in the same reaction vessel and at the same time. Parallel nucleic acid amplification reactions may be conducted, for example, by including reagents used for each nucleic acid amplification reaction in a reaction vessel to obtain a reaction mixture and subjecting the reaction mixture to conditions used for each nucleic amplification reaction. Any suitable number of nucleic acid amplification reactions may be conducted in parallel. In some cases, at least 1, 2, 3, 4, 5, 6, 7, 8, 9, 10, 11, 12, 13, 14, 15, 16, 17, 18, 19, 20, or more nucleic acid amplification reactions are conducted in parallel.

An advantage of conducting nucleic acid amplification reactions in parallel can include multiplexed testing of different but related biomarkers. For example, multiple regions can be tested simultaneously such that a single sample can be used to perform the different assays.

In various aspects, amplified product (e.g., amplified DNA product, amplified RNA) may be detected. The particular type of detection method used may depend, for example, on the particular amplified product, the type of reaction vessel used for amplification, other reagents in a reaction mixture, whether or not a reporter agent was included in a reaction mixture, and if a reporter agent was used, the particular type of reporter agent use. Non-limiting examples of detection methods include optical detection, spectroscopic detection, electrostatic detection, electrochemical detection, and the like.

In any of the various aspects, the detection of amplicons may comprise detecting signals corresponding to the plurality of amplicons. The particular type of detection method used may depend, for example, on the particular amplified product, the type of reaction vessel used for amplification, other reagents in a reaction mixture, whether or not a reporter agent was included in a reaction mixture, and if a reporter agent was used, the particular type of reporter agent use. Non-limiting examples of detection methods include optical detection, spectroscopic detection, electrostatic detection, electrochemical detection, and the like.

In some embodiments, reagents used for conducting nucleic acid amplification, may also include a reporter agent that yields a detectable signal whose presence or absence is indicative of the presence of an amplified product. The intensity of the detectable signal may be proportional to the amount of amplified product. In some cases, where amplified product is generated of a different type of nucleic acid than the nucleic acid sample initially amplified, the intensity of the detectable signal may be proportional to the amount of amplified nucleic acid. The use of a reporter agent also enables real-time amplification methods, including real-time PCR for DNA amplification.

Reporter agents may be linked with nucleic acids, including probes or amplified products, by covalent or non-covalent interactions. Non-limiting examples of non-covalent interactions include ionic interactions, Van der Waals forces, hydrophobic interactions, hydrogen bonding, and combinations thereof. In some embodiments, reporter agents may bind to initial reactants and changes in reporter agent levels may be used to detect amplified product. In some embodiments, reporter agents may only be detectable (or non-detectable) as nucleic acid amplification progresses. In some embodiments, an optically-active dye (e.g., a fluorescent dye) may be used as may be used as a reporter agent. Non-limiting examples of dyes include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, - 83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), 5- (or 6-) iodoacetamidofluorescein, 5-{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, ATTO 390, 425, 465, 488, 495, 520, 532, Rho6G, 550, 565, Rho3B, Rho11, Rho12, Thio12, Rho101, 590, 594, Rho13, 610, 611X, 620, Rho14, 633, 647, 647N, 655, Oxa12, 665,680, 700, 725, 740 or other fluorophores.

In some case, the signals may be radioactive signals. For example, a reporter agent may be a radioactive species. Non-limiting examples of radioactive species include ¹⁴C, ¹²³I, ¹²⁴I, ¹²⁵I, ¹³¹I, ^{99m}Tc, ³⁵S, or ³H. The radioactive species may replace an atom in a probe such that the probe can be detected. The radioactive species may be an atom in a nucleotide in which the nucleotide is incorporated in to an amplicon.

The detection of amplicons may comprise using antibodies. The antibodies may bind a specific sequence or chemical moiety. The chemical moiety may be attached or conjugated to the probe.

The detection of amplicons may comprise using gel electrophoresis to separate the amplicons by size. The size of the amplicon may be correlated with a particular amplicon.

The detection of amplicons may comprise detecting optical signals corresponding to the plurality of amplicons. The detection of amplicons may comprise using the dyes that bind to nucleic acids. The dyes may be non-specific and bind all nucleic acids. In some cases, a reporter agent may be an enzyme that is capable of generating a detectable signal. Detectable signal may be produced by activity of the enzyme with its substrate or a particular substrate in the case the enzyme has multiple substrates. Non-limiting examples of enzymes that may be used as reporter agents include alkaline phosphatase, horseradish peroxidase, I²-galactosidase, alkaline phosphatase, β-galactosidase, acetylcholinesterase, and luciferase. The enzyme may be linked to a probe such that the probe can be detected.

The optical signals may be fluorescent signals. The detection of amplicons may comprise nucleic acid probes which include fluorescent signals. A fluorescent dye or enzyme may be conjugated to an antibody. The enzyme may also generate a fluorescent signal. The fluorescent signal may be generated by a fluorescent or optically active dye. Non-limiting examples of dyes include those described elsewhere herein.

In some examples, the optical signals correspond to wavelengths of light. In some examples, each optical signal corresponds to a different wavelength of light. In some examples, the optical signals may correspond to the same wavelength. The optical signals may correspond to a wavelength or a wavelength range. In some cases, portions of the optical signals may correspond to one wavelength or wavelength range and another portion of the optical signal corresponds to another wavelength or wavelength range

In various aspects, a nucleic acid sample obtained from a subject is amplified. In some embodiments, the nucleic acid sample is obtained from the subject and provided in the single vessel without any filtration, extraction or purification. In some cases, the nucleic acid sample is obtained directly from the subject. A nucleic acid sample obtained directly from a subject generally refers to a nucleic acid sample that has not been further processed after being obtained from the subject, with the exception of any means used to collect the nucleic acid sample from the subject for further processing. For example, blood is obtained directly from a subject by accessing the subject's circulatory system, removing the blood from the subject (e.g., via a needle), and entering the removed blood into a receptacle. The receptacle may comprise reagents (e.g., anti-coagulants) such that the blood sample is useful for further analysis. In another example, a swab may be used to access epithelial cells on an oropharyngeal surface of the subject. After obtaining the nucleic acid sample from the subject, the swab containing the nucleic acid sample can be contacted with a fluid (e.g., a buffer) to collect the fluid from the swab.

In some embodiments, a nucleic acid sample has not been purified when provided in a reaction vessel. In some embodiments, the nucleic acid of a nucleic acid sample has not been extracted when the nucleic acid sample is provided to a reaction vessel. For example, the RNA or DNA in a nucleic acid sample may not be extracted from a biological tissue or cell when providing the nucleic acid sample to a reaction vessel. Moreover, in some embodiments, a nucleic acid sample may not be concentrated prior to being provided to a reaction vessel.

In some embodiments, the nucleic acid sample has been purified in a reaction vessel. The nucleic acid sample may be diluted or concentrated to achieve different concentrations of nucleic acids. The concentration of the nucleic acids in the nucleic acid sample may at least 0.1 nanograms per microliter (ng/μL), 0.2 ng/μL, 0.5 ng/μL, 1 ng/μL, 2 ng/μL, 3 ng/μL, 5 ng/μL, 10 ng/μL, 20 ng/μL, 30 ng/μL, 40, ng/μL, 50 ng/μL, 100 ng/μL, 1000 ng/μL, 10000 ng/μL or more. In some cases, the concentration of the nucleic acids in the nucleic acid sample may be at most ng/μL, 0.2 ng/μL, 0.5 ng/μL, 1 ng/μL, 2 ng/μL, 3 ng/μL, 5 ng/μL, 10 ng/μL, 20 ng/μL, 30 ng/μL, 40, ng/μL, 50 ng/μL, 100 ng/μL, 1000 ng/μL, 10000 ng/μL or less.

A nucleic acid sample may be from any suitable biological matter that comprises nucleic acid obtained from a subject. A nucleic acid sample may be solid matter (e.g., biological tissue) or may be a fluid (e.g., a biological fluid). In general, a biological fluid can include any fluid associated with living organisms. Non-limiting examples of a nucleic acid sample include blood (or components of blood—e.g., white blood cells, red blood cells, platelets) obtained from any anatomical location (e.g., tissue, circulatory system, bone marrow) of a subject, cells obtained from any anatomical location of a subject, skin, heart, lung, kidney, breath, bone marrow, stool, semen, vaginal fluid, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, cavity fluids, sputum, pus, micropiota, meconium, breast milk, prostate, esophagus, thyroid, serum, saliva, urine, gastric and digestive fluid, tears, ocular fluids, sweat, mucus, earwax, oil, glandular secretions, spinal fluid, hair, fingernails, skin cells, plasma, nasal swab or nasopharyngeal wash, spinal fluid, cord blood, emphatic fluids, and/or other excretions or body tissues.

A nucleic acid sample may be obtained from a subject by any means. Non-limiting examples of means to obtain a nucleic acid sample directly from a subject include accessing the circulatory system (e.g., intravenously or intra-arterially via a syringe or other needle), collecting a secreted biological sample (e.g., feces, urine, sputum, saliva, etc.), surgically (e.g., biopsy), swabbing (e.g., buccal swab, oropharyngeal swab), pipetting, and breathing. Moreover, a nucleic acid sample may be obtained from any anatomical part of a subject where a desired biological sample is located.

In various aspects, lysis of a cell or cell derivative may be performed. To perform lysis a lysis agent may be used. In cases where a lysis agent is desired, any suitable lysis agent may be used, including commercially available lysis agents. Non-limiting examples of lysis agents include Tris-HCl, EDTA, detergents (e.g., Triton X-100, SDS), lysozyme, glucolase, proteinase E, viral endolysins, exolysins zymolose, Iyticase, proteinase K, endolysins and exolysins from bacteriophages, endolysins from bacteriophage PM2, endolysins from the B. subtilis bacteriophage PBSX, endolysins from Lactobacillus prophages Lj928, Lj965, bacteriophage 15 Phiadh, endolysin from the Streptococcus pneumoniae bacteriophage Cp-I, bifunctional peptidoglycan lysin of Streptococcus agalactiae bacteriophage B30, endolysins and exolysins from prophage bacteria, endolysins from Listeria bacteriophages, holin-endolysin, cell 20 lysis genes, holWMY Staphylococcus wameri M phage varphiWMY, Iy5WMY of the Staphylococcus wameri M phage varphiWMY, and combinations thereof. In some cases a buffer may comprise a lysis agent (e.g., a lysis buffer). An example of a lysis buffer is sodium hydroxide (NaOH).

In various aspects, a nucleic acid sample is amplified to generate an amplified product. A nucleic acid sample may be a RNA or a DNA. In cases where the nucleic acid sample is a DNA, the DNA may be any type of DNA, including types of DNA described elsewhere herein. The DNA may be genomic DNA. The DNA may be human genomic DNA. The DNA may be a segment of human genomic DNA correlated to a disease state.

In various aspects, the nucleic acid sample is a chromosome or a derivative of the chromosome. The nucleic sample can be processed by enzymes. Processing may include but is not limited to fragmentation of the nucleic acid sample, ligation of additional sequences, binding to other molecules. The processing may improve the accuracy of the various aspects.

In some embodiments, the nucleic acid sample may be associated with a disease. The disease may be spinal muscular atrophy. In some embodiments, the amplification protocol can be directed to assaying for the presence of the disease based on a presence of the amplified product.

In any of the various aspects, the genetic aberration of the SMN1 gene is a two-copy haplotype. The genetic aberration of the SMN1 gene may be a point mutation. The genetic aberration of the SMN1 gene may be a gene conversion to SMN2. The genetic aberration of the SMN1 gene may be a gene deletion of SMN1. The genetic aberration of the SMN1 gene may be a gene duplication. The genetic aberration of the SMN1 gene may be an insertion. The genetic aberration may cause a change in the sequence of SMN1 polypeptide. The genetic aberration of the SMN1 gene may cause a change in the level of SMN1 polypeptide. The genetic aberration of the SMN1 gene may cause a change in the amount of SMN1 polypeptide expressed. The genetic aberration of the SMN1 gene may cause a change in the overall activity of the SMN1 polypeptide. The genetic aberration of the SMN1 gene may on the same chromosome as another genetic aberration.

In any of the various aspects, the first probe may bind to the sequence of the SMN1 gene. The sequence of the first probe may have a sequence homology to exon 7 of SMN1. The sequence of the first probe may have a sequence homology to region c.840 in exon 7 of SMN1. The sequence of first probe may differ from the sequence of the second probe by a single nucleotide. The sequence of first probe may differ from the sequence of the second probe by more than 1, 2, 3, 4, 5, 10, or more nucleotides.

In any of the various aspects, the second probe may bind to the sequence of the SMN2 gene. The sequence of the second probe may have sequence homology to exon 7 of SMN2. The sequence of the second probe may have sequence homology to region c.840 in exon 7 of SMN2.

In any of the various aspects, the third probe binds to the sequence of SMN1 or SMN2. The sequence of the third probe may have sequence homology to exon 8 of SMN1 or SMN2. The sequence of the third probe may have sequence homology to region g.22706_22707 of the human chromosome. The sequence of third probe may differ from the sequence of the fourth probe by a single nucleotide. The sequence of third probe may differ from the sequence of the fourth probe by 2 nucleotides. The sequence of the third probe may differ from the sequence of the fourth probe by more than 1, 2, 3, 4, 5, 10, or more nucleotides.

In any of the various aspects, the forth probe may bind to the sequence of SMN1 or SMN2. The sequence of the fourth probe may have sequence homology to exon 8 of SMN1 or SMN2. The sequence of the fourth probe may have sequence homology to region g.22706_22707 of the human chromosome. The sequence of the fourth probe may have sequence homology to region g.22706_22707delAT of the human chromosome.

In any of the various aspects, the probe set further comprises a fifth probe that is configured to provide a control signal. The fifth probe may bind to a sequence that does not correspond to genes or sequences associated with SMA. The fifth probe may bind to a third locus. The fifth probe may bind to the RPP30 gene. The fifth probe may allow monitoring of gene copy number. The fifth probe may be used to calculate a gene copy number. The fifth probe may be used to calculate a copy number of SMN1. The fifth probe may be used to calculate a copy number of SMN2. The fifth probe may be used to calculate a copy number of SMN1. The fifth probe may be used to calculate a copy number of SMN2. The fifth probe may be used to check that the reaction is properly executed. The fifth probe may be used to eliminate false negatives. In some embodiments, the control signal is an optical signal. The control signal may be used to compare to signals for other probes in the probe set. Comparing the intensity of the control signal to the intensity of signals from other probes may allow copy number in a gene to be identified. Additionally, comparing the intensity of the control signal to the intensity of signals from other probes may be used to determine that the proper reaction has taken place.

In any of the various aspects, the first probe, second probe, third probe and fourth probe are quantitative polymerase chain reaction probes. In some embodiments, the probe is optically active when hybridized with an amplified product. Due to sequence-specific binding of the probe to the amplified product, use of probes can increase specificity and sensitivity of detection. A probe may be linked to any of the optically-active reporter agents (e.g., dyes) described herein and may also include a quencher capable of blocking the optical activity of an associated dye. Non-limiting examples of probes include TaqMan™ probes, LNA labeled probes, TaqMan Tamara™ probes, TaqMan MGB™ probes, or Lion probes.

In some embodiments, the first probe, second probe, third probe and fourth probe are hydrolysis probes. In some cases, at least one of the first probe, second probe, third probe and fourth probe can be a hydrolysis probe. In some cases, at least two of the first probe, second probe, third probe and fourth probe can be hydrolysis probes. In some cases, at least three of the first probe, second probe, third probe and fourth probe can be hydrolysis probes. In some embodiments, the hydrolysis probes are TaqMan probes. A TaqMan probe includes, for example, a quencher linked at one end of an oligonucleotide. At the other end of the oligonucleotide is an optically active dye, such as, for example, a fluorescent dye. The optically-active dye and quencher are in close enough proximity such that the quencher is capable of blocking the optical activity of the dye. Upon hybridization with amplified product, the dye and quencher are still in close enough proximity such that the quencher is capable of blocking the optical activity of the dye. During the amplification reaction, the probe is degraded by the exonuclease activity of the DNA polymerase and releases the dye. As the dye is no longer in close enough proximity to the quencher to block optical activity, the dye emits a detectable signal.

In any of the various aspects, the first probe, second probe, third probe and fourth probe are molecular beacons. In some embodiments, at least one of the first probe, second probe, third probe and fourth probe is a molecular beacon. In some embodiments, at least two of the first probe, second probe, third probe and fourth probe are molecular beacons. In some embodiments, at least three of the first probe, second probe, third probe and fourth probe are molecular beacons. A molecular beacon includes, for example, a quencher linked at one end of an oligonucleotide in a hairpin conformation. At the other end of the oligonucleotide is an optically active dye, such as, for example, a fluorescent dye. In the hairpin configuration, the optically-active dye and quencher are brought in close enough proximity such that the quencher is capable of blocking the optical activity of the dye. Upon hybridizing with amplified product, however, the oligonucleotide assumes a linear conformation and hybridizes with a target sequence on the amplified product. Linearization of the oligonucleotide results in separation of the optically-active dye and quencher, such that the optical activity is restored and can be detected. The sequence specificity of the molecular beacon for a target sequence on the amplified product can improve specificity and sensitivity of detection.

In any of the various aspects, the first probe, second probe, third probe and fourth probe are primers for performing nucleic acid amplification reactions at the first locus and the second locus. In some embodiments, at least one of the first probe, second probe, third probe and fourth probe are primers for performing nucleic acid amplification reactions at the first locus or the second locus. In some embodiments, at least two of the first probe, second probe, third probe and fourth probe are primers for performing nucleic acid amplification reactions at the first locus or the second locus. In some embodiments, at least three of the first probe, second probe, third probe and fourth probe are primers for performing nucleic acid amplification reactions at the first locus or the second locus.

In any of the various aspects, the first probe, second probe, third probe and fourth probe are configured to emit different signals. In some embodiments, the different signals are different optical signals. In some cases, the different optical signals are different enough as to be resolvable by a detector. When the data for the detector is analyzed, the different optical signals are able to be distinguishable and the probes from which the optical signal was generated from can be identified. In some cases, the optical signal of a probe can be identified by the human eye, without the help of a detector.

In any of the various aspects, at least one of the first probe, second probe, third probe and fourth probe comprises at least one locked nucleic acid. In some embodiments, at least one of the first probe, second probe, third probe and fourth probe comprises at least two locked nucleic acids. In some embodiments, at least one of the first probe, second probe, third probe and fourth probe comprises at least three locked nucleic acids. In some embodiments, at least one of the first probe, second probe, third probe and fourth probe comprises at least four locked nucleic acids. In some embodiments, at least one of the first probe, second probe, third probe and fourth probe comprises at least five locked nucleic acids. In some embodiments, at least one of the first probe, second probe, third probe and fourth probe comprises at least six locked nucleic acids. The locked nucleic acids can be at a specific position within the probe. The position of the locked nucleic acids changes the affinity of the probe to its target. In some cases, at least one of the first probe, second probe, third probe and fourth probe may include at least 1, 2, 3, 4, 5, 6, 7, 8, 9, 10, 12, 14, 16, 18, 20, 30, 40, 50, 60, 70, 80, 90, 100, or more locked nucleic acids. In some cases, a probe may include at most 1, 2, 3, 4, 5, 6, 7, 8, 9, 10, 12, 14, 16, 18, 20, 30, 40, 50, 60, 70, 80, 90, 100, or less locked nucleic acids.

In any of the various aspects, each of at least two of the first probe, second probe, third probe and fourth probe comprises at least one locked nucleic acid. In some embodiments, each of at least three of the first probe, second probe, third probe and fourth probe comprises at least one locked nucleic acid. In some embodiments each of the first probe, second probe, third probe and fourth probe comprises at least one locked nucleic acid.

In some embodiments, the probes compete with one another for binding sites. The competing for binding can increase the overall specificity of the probes. The probes can hybridize to an amplified product in which a probe is substantially complementary to the amplified product. A probe that is completely complementary to an amplified product may have a higher affinity to the amplified product than a probe that is substantially complementary. When at least two probes are allowed to hybridize with amplified products that comprise sequences that are completely complementary to the at least two probes and substantially complementary to the at least two probes, the probes may compete with one another for the completely complementary sequence resulting in both probes binding to the completely complementary sequence and not the substantially complementary sequence.

In some embodiments, the probes competing with one another for binding sites reduces background signal and increases the accuracy. For example, due to genomic sequence similarity of the SMN1 and SMN2 gene, a probe completely complementary to a sequence of SMN1 can bind to a similar but not identical sequence on the SMN2 gene. However when a probe to the SMN2 gene is introduced, the completely complementary probe to the SMN2 sequence may bind to the SMN2 sequence and prevent the completely complementary probe to the SMN1 sequence from binding to the SMN2 sequence. When the SMN1 sequence is present in a reduced number of copies, the completely complementary SMN1 probe may be prone to bind to the SMN2 sequence. When a completely complementary SMN2 probe with higher affinity to the SMN2 sequence is present, it competes with the completely complementary SMN1 probe in binding to the SMN2 sequence thus preventing the hydrolysis of the SMN1 probe and generation of a nonspecific signal. The same effect is generated for the probe to the SMN2. In some embodiments, at least two probes compete with one another for binding. In some embodiments, at least three probes compete with one another for binding. In some embodiments, at least four probes compete with one another for binding. In some embodiments, at least five probes compete with one another for binding.

In any of the various aspects, the probes are lyophilized. The first probe, second probe, third probe and fourth probe may be lyophilized together in a single vessel. The first probe, second probe, third probe and fourth probe may be lyophilized individually. The fifth probe may be lyophilized with the first probe, second probe, third probe and fourth probe in a single vessel. The fifth probe may also be lyophilized individually. The probes may be lyophilized in a combination of individually and together. For example, some probes may be lyophilized together with other probes, and other probes are lyophilized individually. After lyophilization, the probes can be re-hydrated for use. The rehydration may be done with a diluent containing buffers, bacterial inhibitors and DNA reagent grade water to ensure quality of the reaction. In some cases, when the probes are lyophilized in different vessels, the probes can be rehydrated and then mixed together in a single vessel. The probes can be lyophilized along with enzymes, nucleotides, buffers, salts, sugars, primers, or a combination thereof. Alternatively, the enzymes, nucleotides, or primers may not be lyophilized along with the probes and are added to the reaction vessel prior to the reaction.

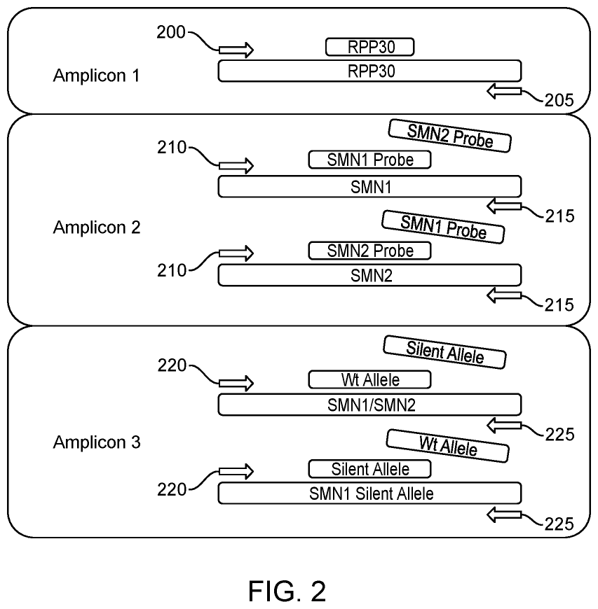

FIG. 2 illustrates an example embodiment of the probe set, primers, and amplicons used to detect copy number of SMN1, SMN2 and the presence of the two-copy haplotype. Three separate amplicons are produced in the reaction, one corresponding to a control, one corresponding to SMN1 and/or SMN2, and one corresponding to the detection of the two-copy haplotype. Amplicon 1 is the control amplicon. This amplicon is created with primers 200 and 205 to a gene that has a copy number that is constant across all subjects. In this embodiment, that gene is RPP30. Candidate genes for this amplicon may include those whose loss results in embryonic lethality. Amplicon 2 may correspond to SMN1 gene and SMN2 gene. PCR primers 210 and 215 correspond to regions of the SMN1 and SMN2 gene which are identical or near identical to one another allow the PCR to amplify both SMN1 and SMN2 genes, resulting in two different versions of Amplicon 2, labeled as SMN1 and SMN2. Amplicon 3 contains a sequence correlated to the two-copy haplotype labeled as an SMN1 silent allele or the wild type SMN1 or SMN2 allele labeled as SMN1/SMN2. This amplicon is produced by primers 220 and 225 flanking the sequence correlated to the two-copy haplotype. Probes RPP30, SMN1 Probe, SMN2 Probe, WT allele, and Silent Allele are added into the reaction mixture. Probe RPP30 has affinity to the RPP30 gene of Amplicon 1 to allow detection of a base line copy number. Probe SMN1 Probe has highest affinity to the SMN1 version of Amplicon 2 compared to the other probes in the reaction. Probe SMN2 Probe has highest to the SMN2 version of Amplicon 2 affinity compared to the other probes in the reaction. Although the SMN1 and SMN2 version of Amplicon 2 are similar in sequence, probes 220 and 225 are specific to their respective amplicon version with little or no cross binding when both the SMN1 Probe and SMN2 Probe are present. This is illustrated in the figure with the SMN2 Probe having a general proximity to the SMN1 but is not directly adjacent. The same is shown for the SMN1 Probe and the SMN2. Probe WT Allele has affinity to SMN1/SMN2 version of Amplicon 3. Probe Silent Allele has affinity to the SMN1 Silent Allele version of Amplicon 3. Although amplicon SMN1 Silent Allele and WT Allele are similar in sequence, probes WT Allele and SMN1 Silent Allele are specific to their respective amplicon version with little or no cross binding when both the WT Allele and SMN1 Silent Allele probes are present. Each probe may be attached to a different dye or other molecule resulting in a signal, such that each probe when in one reaction vessel can be independently measured.

In various aspects of the present disclosure, a vessel may be used to perform a reaction. In an example, the reaction is completed in a single vessel. The reaction vessel may contain probes, primers, nucleotides, and enzymes to allow for an amplification reaction to be performed. A biological sample may be added to a reaction vessel to perform an amplification reaction. Any suitable reaction vessel may be used. In some embodiments, a reaction vessel comprises a body that can include an interior surface, an exterior surface, an open end, and an opposing closed end. In some embodiments, a reaction vessel may comprise a cap. The cap may be configured to contact the body at its open end, such that when contact is made the open end of the reaction vessel is closed. In some cases, the cap is permanently associated with the reaction vessel such that it remains attached to the reaction vessel in open and closed configurations. In some cases, the cap is removable, such that when the reaction vessel is open, the cap is separated from the reaction vessel. In some embodiments, a reaction vessel may be sealed, optionally hermetically sealed.

A reaction vessel may be of varied size, shape, weight, and configuration. In some examples, a reaction vessel may be round or oval tubular shaped. In some embodiments, a reaction vessel may be rectangular, square, diamond, circular, elliptical, or triangular shaped. A reaction vessel may be regularly shaped or irregularly shaped. In some embodiments, the closed end of a reaction vessel may have a tapered, rounded, or flat surface. Non-limiting examples of types of a reaction vessel include a tube, a well, a capillary tube, a cartridge, a cuvette, a centrifuge tube, or a pipette tip. Reaction vessels may be constructed of any suitable material with non-limiting examples of such materials that include glasses, metals, plastics, and combinations thereof.

In some embodiments, a reaction vessel is part of an array of reaction vessels. An array of reaction vessels may be particularly useful for automating methods and/or simultaneously processing multiple samples. For example, a reaction vessel may be a well of a microwell plate comprised of a number of wells. In another example, a reaction vessel may be held in a well of a thermal block of a thermocycler, in which the block of the thermal cycle comprises multiple wells each capable of receiving a sample vessel. An array comprised of reaction vessels may comprise any appropriate number of reaction vessels. For example, an array may comprise at least 2, 4, 6, 8, 10, 12, 14, 16, 18, 20, 25, 35, 48, 96, 144, 384, or more reaction vessels. A reaction vessel part of an array of reaction vessels may also be individually addressable by a fluid handling device, such that the fluid handling device can correctly identify a reaction vessel and dispense appropriate fluid materials into the reaction vessel. Fluid handling devices may be useful in automating the addition of fluid materials to reaction vessels.

In some embodiments, information regarding the presence of and/or an amount of amplified product (e.g., amplified DNA product) may be outputted to a recipient. Information regarding amplified product may be outputted via any suitable means. In some embodiments, such information may be provided verbally to a recipient. In some embodiments, such information may be provided in a report. A report may include any number of desired elements, with non-limiting examples that include information regarding the subject (e.g., sex, age, race, health status, etc.) raw data, processed data (e.g. graphical displays (e.g., figures, charts, data tables, data summaries), determined cycle threshold values, calculation of starting amount of target polynucleotide), conclusions about the presence of the target nucleic acid, identification information, diagnosis information, prognosis information, disease information, and the like, and combinations thereof. The report may be provided as a printed report (e.g., a hard copy) or may be provided as an electronic report. In some embodiments, including cases where an electronic report is provided, such information may be outputted via an electronic display (e.g., an electronic display screen), such as a monitor or television, a screen operatively linked with a unit used to obtain the amplified product, a tablet computer screen, a mobile device screen, and the like. Both printed and electronic reports may be stored in files or in databases, respectively, such that they are accessible for comparison with future reports.

Moreover, a report may be transmitted to the recipient at a local or remote location using any suitable communication medium including, for example, a network connection, a wireless connection, or an internet connection. In some embodiments, a report can be sent to a recipient's device, such as a personal computer, phone, tablet, or other device. The report may be viewed online, saved on the recipient's device, or printed. A report can also be transmitted by any other suitable means for transmitting information, with non-limiting examples that include mailing a hard-copy report for reception and/or for review by a recipient.

Moreover, such information may be outputted to various types of recipients. Non-limiting examples of such recipients include the subject from which the biological sample was obtained, a physician, a physician treating the subject, a clinical monitor for a clinical trial, a nurse, a researcher, a laboratory technician, a representative of a pharmaceutical company, a health care company, a biotechnology company, a hospital, a human aid organization, a health care manager, an electronic system (e.g., one or more computers and/or one or more computer servers storing, for example, a subject's medical records), a public health worker, other medical personnel, and other medical facilities.

In various aspects, the systems may include an electronic display screen. An electronic display screen may be used display information relating to performing a method described herein. An electronic display screen may be used display data obtained from performing a method described herein. The electronic display screen may have a user interface that displays a graphical element that is accessible by a user to execute an amplification protocol to amplify nucleic acid sample. A computer processor (including any suitable device having a computer processor as described elsewhere herein) may be coupled to the electronic display screen and programmed to execute the amplification protocol upon selection of the graphical element by the user. The amplification protocol can comprise subjecting a reaction mixture comprising the nucleic acid sample and reagents used for conducting nucleic acid amplification to a plurality of series of primer extension reactions to generate amplified product. Moreover, each series of primer extension reactions can comprise two or more cycles of incubating the reaction mixture under a denaturing condition that is characterized by a denaturing temperature and a denaturing duration, followed by incubating the reaction mixture under an elongation condition that is characterized by an elongation temperature and an elongation duration. An individual series can differ from at least one other individual series of the plurality with respect to the denaturing condition and/or the elongation condition.

In some cases, a user interface can be a graphical user interface. Moreover, a user interface can include one or more graphical elements. Graphical elements can include image and/or textual information, such as pictures, icons and text. The graphical elements can have various sizes and orientations on the user interface. Furthermore, an electronic display screen may be any suitable electronic display including examples described elsewhere herein. Non-limiting examples of electronic display screens include a monitor, a mobile device screen, a laptop computer screen, a television, a portable video game system screen and a calculator screen. In some embodiments, an electronic display screen may include a touch screen (e.g., a capacitive or resistive touch screen) such that graphical elements displayed on a user interface of the electronic display screen can be selected via user touch with the electronic display screen.

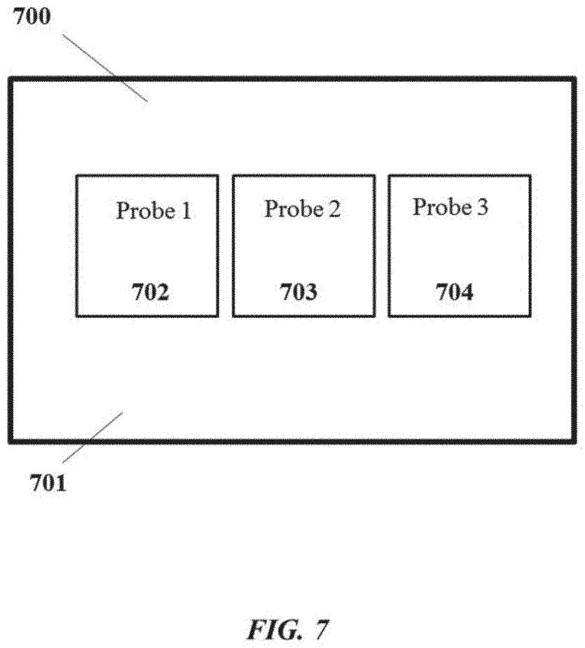

In some embodiments, the user interface displays a plurality of graphical elements. Each of the graphical elements can be associated with a given amplification protocol among a plurality of amplification protocols. Each of the plurality of amplification protocols may include a different combination of series of primer extension reaction. In some cases, though, a user interface may display a plurality of graphical elements associated with the same amplification protocol. In some cases, each of the graphical elements can be associated with a plurality of probes. In some cases, each of the graphical elements can be associated with a given probe among a plurality of probes. An example of a user interface having a plurality of graphical elements each associated with a given probe or probe signal is shown in FIG. 7. As shown in FIG. 7, an example electronic display screen 700 associated with a computer processor includes a user interface 701. The user interface 701 includes a display of graphical elements 702, 703 and 704. Each of the graphical elements can be associated with a probe or probe signal (e.g., “Probe. 1” for graphical element 702, “Probe 2” for graphical element 703 and “Probe 3” for graphical element 704). Upon user selection (e.g., user touch when the electronic display screen 700 includes a touch-screen having the user interface) of particular graphical element, the particular data associated to a probe or probe signal associated with the graphical element can graphed and displayed by an associated computer processor. For example, when a user selects graphical element 703, data corresponding to “Probe 2” is graphed and displayed by the associated computer processor. Where only three graphical elements are shown in the example user interface 701 of FIG. 7, a user interface may have any suitable number of graphical elements. Moreover, where each graphical element shown in the user interface 701 of FIG. 7 is associated with only one probe or probe signal, each graphical element of a user interface can be associated with one or more probes or probe signals (e.g., a series of different sample containing the same probe sequence) such that an associated computer processor graphs and displays the probe signal for a series of different samples upon user interaction with the graphical element.

In various aspects, the system may include an input module that receives a user request to amplify a nucleic acid present in a nucleic acid sample obtained from a subject. An input module may also be used to perform steps relating to a method described herein. Any suitable module capable of accepting such a user request may be used. The input module may comprise, for example, a device that comprises one or more processors. Non-limiting examples of devices that comprise processors (e.g., computer processors) include a desktop computer, a laptop computer, a tablet computer (e.g., Apple® iPad, Samsung® Galaxy Tab), a cell phone, a smart phone (e.g., Apple® iPhone, Android® enabled phone), a personal digital assistant (PDA), a video-game console, a television, a music playback device (e.g., Apple® iPod), a video playback device, a pager, and a calculator. The one or more processors may be associated with one or more controllers, calculation units, and/or other units of a computer system, or implanted in firmware as desired. If implemented in software, the routines (or programs) may be stored in any computer readable memory such as in RAM, ROM, flash memory, a magnetic disk, a laser disk, or other storage medium. The one or more processors may be a single processor (e.g., single core or multi-core processor). The one or more processors may be a plurality of processors (e.g., single core or multi-core processors). Likewise, this software may be delivered to a device via any delivery method including, for example, over a communication channel such as a telephone line, the internet, a local intranet, a wireless connection, etc., or via a transportable medium, such as a computer readable disk, flash drive, etc. The various steps may be implemented as various blocks, operations, tools, modules or techniques which, in turn, may be implemented in hardware, firmware, software, or any combination thereof. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

In some embodiments, the input module is configured to receive a user request to perform amplification of the target nucleic acid. The input module may receive the user request directly (e.g. by way of an input device such as a keyboard, mouse, or touch screen operated by the user) or indirectly (e.g. through a wired or wireless connection, including over the internet). Via output electronics, the input module may provide the user's request to the amplification module. In some embodiments, an input module may include a user interface (UI), such as a graphical user interface (GUI), that is configured to enable a user provide a request to amplify the target nucleic acid. A GUI can include textual, graphical and/or audio components. A GUI can be provided on an electronic display, including the display of a device comprising a computer processor. Such a display may include a resistive or capacitive touch screen.

Non-limiting examples of users include the subject from which the biological sample was obtained, medical personnel, clinicians (e.g., doctors, nurses, laboratory technicians), laboratory personnel (e.g., hospital laboratory technicians, research scientists, pharmaceutical scientists), a clinical monitor for a clinical trial, or others in the health care industry.

In various aspects, the system may include an amplification module for performing nucleic acid amplification reaction on target nucleic acid or a portion thereof. The amplification module may respond to a user request received by the input module. The amplification module may be capable of executing any of the methods described herein and may include any of a fluid handling device, one or more thermocyclers, means for receiving one or more reaction vessels (e.g., wells of a thermal block of a thermocycler), a detector (e.g., optical detector, spectroscopic detector, electrochemical detector) capable of detecting amplified product, and means for outputting information (e.g., raw data, processed data, or any other type of information described herein) regarding the presence and/or amount of amplified product (e.g., amplified DNA product) to a recipient. In some cases, the amplification module may comprise a device with a computer processor as described elsewhere herein and may also be capable of analyzing raw data from detection, with the aid of appropriate software. Moreover, in some embodiments, the amplification module may comprise input electronics used to receive instructions from the input module and may comprise output electronics used to communicate with the output module.

In some embodiments, one or more steps of providing materials to a reaction vessel, amplification of nucleic acids, detection of amplified product, and outputting information may be automated by the amplification module. In some embodiments, automation may comprise the use of one or more fluid handlers and associated software. Several commercially available fluid handling systems can be utilized to run the automation of such processes. Non-limiting examples of such fluid handlers include fluid handlers from Perkin-Elmer, Caliper Life Sciences, Tecan, Eppendorf, Apricot Design, and Velocity 11.

In some embodiments, an amplification module may include a real-time detection instrument. Non-limiting examples of such instruments include a real-time PCR thermocycler, QuantStudio 7 Flex Real-Time PCR System, Qiagen Rotogene, Bio Rad CFX systems, ABI PRISM® 7000 Sequence Detection System, ABI PRISM® 7700 Sequence Detection System, Applied Biosystems 7300 Real-Time PCR System, Applied Biosystems 7500 Real-Time PCR System, Applied Biosystems 7900 HT Fast Real-Time PCR System (all from Applied Biosystems); LightCycler™ System (Roche Diagnostics GmbH); Mx3000P™ Real-Time PCR System, Mx3005P™ Real-Time PCR System, and Mx4000® Multiplex Quantitative PCR System (Stratagene, La Jolla, Calif.); and Smart Cycler System (Cepheid,). In some embodiments, an amplification module may comprise another automated instrument such as, for example, a COBAS® AmpliPrep/COBAS® TaqMan® system (Roche Molecular Systems), a TIGRIS DTS system (Hologic Gen-Probe, San Diego, Calif.), a PANTHER system (Hologic Gen-Probe, San Diego, Calif.), a BD MAX™ system (Becton Dickinson), a GeneXpert System (Cepheid/Danaher), a Filmarray® (BioFire Diagnostics) system, an iCubate system, an IDBox system (Luminex), an EncompassMDx™ (Rheonix) system, a Liat™ Aanlyzer (IQuum) system, a Biocartis' Molecular Diagnostic Platform system, an Enigma® ML system (Enigma Diagnostics), a T2Dx® system (T2 Biosystems), a Verigene® system (NanoSphere), a Great Basin's Diagnostic System, a Unyvero™ System (Curetis), a PanNAT system (Micronics), a Spartan™ RX system (Spartan Bioscience) QuantStudio 3D Digital PCR System, or a QX200 Droplet Digital PCR System.

As an alternative, a nucleic acid sample of a subject may be processed with kits and methods of the present disclosure and subjected to sequencing to identify whether the subject has SMA or is a carrier of SMA. Such sequencing may be next generation sequencing, massively parallel array sequencing (e.g., Illumina), whole genome sequencing, targeted sequencing (e.g., enrichment followed by massively parallel array sequencing), or nanopore based sequencing (e.g., Roche/Genia or Oxford Nanopore). In some examples, kits and probes of the present disclosure are used to generate sequencing libraries that may be subsequently sequenced to generate sequencing reads, which may be analyzed to identify whether the subject has SMA or is a carrier of SMA.

In various aspects, an output module is operatively connected to the amplification module. In some embodiments the output module may comprise a device with a processor as described above for the input module. The output module may include input devices as described herein and/or may comprise input electronics for communication with the amplification module. In some embodiments, the output module may be an electronic display, in some cases the electronic display comprising a UI. In some embodiments, the output module is a communication interface operatively coupled to a computer network such as, for example, the internet. In some embodiments, the output module may transmit information to a recipient at a local or remote location using any suitable communication medium, including a computer network, a wireless network, a local intranet, or the internet. In some embodiments, the output module is capable of analyzing data received from the amplification module. In some cases, the output module includes a report generator capable of generating a report and transmitting the report to a recipient. The report may contain any information regarding the amount and/or presence of amplified product as described elsewhere herein. In some embodiments, the output module may transmit information automatically in response to information received from the amplification module, such as in the form of raw data or data analysis performed by software included in the amplification module. Alternatively, the output module may transmit information after receiving instructions from a user. Information transmitted by the output module may be viewed electronically or printed from a printer.

One or more of the input module, amplification module, and output module may be contained within the same device or may comprise one or more of the same components. For example, an amplification module may also comprise an input module, an output module, or both. In other examples, a device comprising a processor may be included in both the input module and the output module. A user may use the device to request that a target nucleic acid be amplified and may also be used as a means to transmit information regarding amplified product to a recipient. In some cases, a device comprising a processor may be included in all three modules, such that the device comprising a processor may also be used to control, provide instructions to, and receive information back from instrumentation (e.g., a thermocycler, a detector, a fluid handling device) included in the amplification module or any other module.

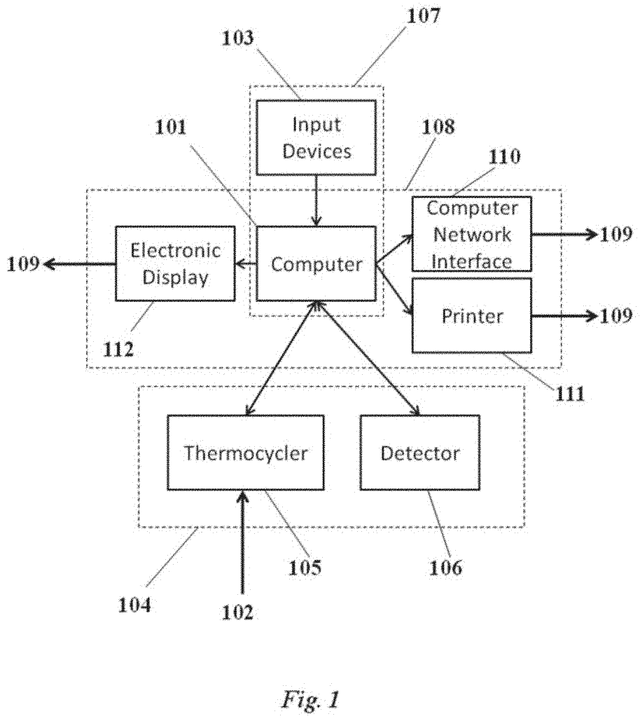

An example system for amplifying a target nucleic acid according to methods described herein is depicted in FIG. 1. The system comprises a computer 101 that may serve as part of both the input and output modules. A user enters a reaction vessel 102 comprising a reaction mixture ready for nucleic acid amplification into the amplification module 104. The amplification module comprises a thermocycler 105 and a detector 106. The input module 107 comprises computer 101 and associated input devices 103 (e.g., keyboard, mouse, etc.) that can receive the user's request to amplify a target nucleic acid in the reaction mixture. The input module 107 communicates the user's request to the amplification module 104 and nucleic acid amplification commences in the thermocycler 105. As amplification proceeds, the detector 106 of the amplification module detects amplified product. Information (e.g., raw data obtained by the detector) regarding the amplified product is transmitted from the detector 106 back to the computer 101, which also serves as a component of the output module 108. The computer 101 receives the information from the amplification module 104, performs any additional manipulations to the information, and then generates a report containing the processed information. Once the report is generated, the computer 101 then transmits the report to its end recipient 109 over a computer network (e.g., an intranet, the internet) via computer network interface 110, in hard copy format via printer 111, or via the electronic display 112 operatively linked to computer 101. In some cases, the electronic display 112

Computer readable medium may take many forms, including but not limited to, a tangible (or non-transitory) storage medium, a carrier wave medium, or physical transmission medium. Non-volatile storage media include, for example, optical or magnetic disks, such as any of the storage devices in any computer(s) or the like, such as may be used to implement the calculation steps, processing steps, etc. Volatile storage media include dynamic memory, such as main memory of a computer. Tangible transmission media include coaxial cables; copper wire and fiber optics, including the wires that comprise a bus within a computer system. Carrier-wave transmission media can take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media may be involved in carrying one or more sequences of one or more instructions to a processor for execution.

### EXAMPLES

### Example 1: Amplification and Detection of Nucleic Acids in Human Cell Sample

Amplification and detection experiments are performed to compare results obtained from nucleic acid samples obtained from healthy individuals and individuals suspected of having spinal muscular atrophy (SMA) or being an SMA carrier. The nucleic acid samples comprising genomic DNA are subject to amplification conditions in the presence of multiple probes, such that area of DNA that correlated to SMA is amplified. Each nucleic acid sample is obtained directly from a subject via an oropharyngeal swab. In each experimental set, a negative control (e.g., a sample comprising no genomic DNA) is also subject to amplification. Five microliters of each sample are combined in a 25 microliter (μL) reaction tube with reagents used for amplification and detection of DNA. The reagents used to conduct the DNA amplification and qPCR are supplied as a commercially available pre-mixture (a DNA Polymerase (e.g., HotStarTaq® DNA Polymerase)), and dNTPs. Moreover, the reaction tubes also include a TaqMan™ probe comprising a FAM dye for detection of amplified DNA product. To generate amplified DNA product, each reaction mixture is incubated according to a protocol of denaturing and elongation conditions comprising 5 min at 95° C., followed by 20 min at 45° C., followed by 2 min at 95° C., and followed by 40 cycles of 5 seconds at 95° C. and 30 seconds at 55° C. in a real-time PCR thermocycler. Detection of amplified product occurs during incubations. Recorded fluorescence of the FAM dye is plotted against the number of cycles.

### Example 2. Detection of Copy Number of SMN1 and SMN2 and Two-Copy Haplotype Using a Kit

A nucleic acid sample is isolated from the subject and is quantified. The nucleic concentration is then normalized so that 2.5 microliters (μL) of a 2 nanogram per microliter (ng/μL) is used for each reaction in the assay. A first tube is supplied and contains lyophilized products. The tube (denoted for this example as tube A) is briefly centrifuged to allow for lyophilize product to settle to the bottom of the tube. In this example of the kit, the lyophilized product includes probes for SMN1 and SMN2 which correspond to a first locus and are used to determine copy number, a wild type SMN1/SMN2 probe and a two-copy haplotype probe which correspond to a second locus, and a copy number control probe which corresponds to a third locus, totaling five probes. The five probes supplied in this example are the probes have the probe sequences indicated in Table 1. The probes include Locked nucleic acids (LNAs) which are denoted in Table 1 with a “+”. The nucleotide that follows the “+” is a locked nucleic acid. The Table also denotes amplicons that are generated and the respective probes that hybridize to the amplicon. Amplicon 1 is a control amplicon that the control probe binds to. Amplicon 2 is a copy number amplicon. Probes that are used to determine copy number bind to Amplicon 2. Amplicon 3 is the two-copy haplotype probe and probes used to determine the presence of the two-copy haplotype bind to Amplicon 3

TABLE 1
Probes for used to detect SMA

| | qPCR PROBES for SMA | | |
|---|---|---|---|
| | Probe name | Sequence | SEQ ID NO: |
| Amplicon 1 | Copy # Control | TTCTGACCTGAAGGCTCTGCGCG | 1 |
| Amplicon 2 | Copy # Probe | TTA + CAG GGT T + T + C A + GA + CAAA AAT | 2 |
| | for SMN1 | | |
| | Copy # Probe | CTT A + CA + GGG TT + T + TA + G A + CA AAA T | 3 |
| | for SMN2 | | |
| Amplicon 3 | SMN1_Wild Type | CTG GA + C T + CT + A + TT TT + G AAA AA + C CA | 4 |
| | 2-0 Silent allele | | |
| | 2-0 Silent allele | CTG GA + C T + CT + TTT + GAA AAA + CCA | 5 |

Additionally, in this example, the lyophilized product includes enzymes, nucleotides, and primers for the qPCR reaction to occur. The sequences of the primers are in Table 2. Table 2 denotes which amplicon is generated by which primers.

TABLE 2
Primers for amplifying target nucleic acids for detecting SMA
PRIMERS

| | Sequence Site | Direction | Sequence | SEQ ID NO: |
|---|---|---|---|---|
| Amplicon 1 | Control RPP30 | Forward | GCGGTGTTTGCAGATTTGGAC | 6 |
| | Control RPP30 | Reverse | CTCCGGAGTSTGGCCCGA | 7 |
| Amplicon 2 | CNV-c.840C > | Forward | AATGCTTTTTAACATCCATATAAAGCTATCTATAT | 8 |
| | T exon 7 | | | |
| | CNV-c.840C > | Reverse | TGCTGGCAGACTTACTCYTTAATTTAAG | 9 |
| | T exon 7 | | | |
| Amplicon 3 | 2-1 Allele- | Forward | ATTAAAAGTTATGTAATAACCARATGCAATGT | 10 |
| | g.22706_22707delAT | | | |
| | 2-1 Allele- | Reverse | ATCCAATATCATTCAAAATCTAATCCACATTCAAA | 11 |
| | g.22706_22707delAT | | | |

A second tube is supplied containing a diluent. The second tube (denoted for this example as Tube B) are vortexed for 5 seconds and then centrifuged briefly to ensure the contents of the tube are on the bottom of the tube. 1.35 milliliters (mL) of the diluent (from Tube B) are added in to the tube containing the lyophilized product (Tube A). Tube A, containing 1.35 mL of diluent with the lyophilized product, is vortexed for 5 second and then is briefly centrifuged. A resulting master mix solution is made contained the rehydrated probes and reagents for the qPCR reaction.

The master mix is then added in to reaction wells according to Table 3. Nucleic acid samples or template nucleic acids are also added to the reaction wells according to the provided table. As denoted by the table, the user when running the reaction in 48 wells, may pipette 22.5 μL of master mix and 2.5 μL of the 2 ng/μL nucleic acid sample into each well.

TABLE 3
Master mix and template volumes for running qPCR

| Component | 48 wells | 96 wells | 384 wells |
|---|---|---|---|
| Master Mix | 22.5 | 22.5 | 18 |
| Template | 2.5 | 2.5 | 2 |
| Total Volume | 25 μL | 25 μL | 20 μL |

Template nucleic acids may also be control molecules which are recommended to be run at the same time as the nucleic acid samples. Controls containing DNA corresponding to different numbers of SMN1 and SMN2 gene copies, as well as DNA with or without the two copy haplotype may be supplied or obtained otherwise. Possible controls are denoted in Table 4 below. Additionally, a reaction may be run with no template nucleic acid or nucleic acid sample to act as a negative control.

TABLE 4
Control Samples

Copy Number

| Sample ID | SMN1 | SMN2 | Two copy haplotype |
|---|---|---|---|
| NA10684 | 0 | 2 | Absent |
| NA23687 | 1 | 2 | Absent |
| NA12878 | 2 | 2 | Absent |
| NA20359 | 3 | 2 | Present - |
| | | | heterozygous. |

To run the PCR, the PCR plate or tube(s) is placed into the qPCR cycler. For this example, the software is set to analyze the data using Comparative Ct. The target reporter fluorescence is set to those corresponding to the probes. For this example, FAM, ATTO 532, Texas Red, ATTO 550, and ATTO 647 are used with each dye corresponding to one probe. The thermocycler is then run using the thermocycling protocol of Table 5.

TABLE 5
qPCR thermocycler protocol

| Step | Temp. | Time | Cycle |
|---|---|---|---|
| 1 | 95° C. | 30 seconds | 1 cycle |
| 2 | 96° C. | 15 seconds | 45 cycles |
| | 65° C. | 1 minute | |
| | | (Data Collection) | |

After the PCR is run, analysis of the run is performed. A software is used to set a threshold and baseline for each fluorescence signal. The data is then exported as an Excel file for processing. A Macro that is supplied is then applied to the exported data and reports the copy number of SMN1 and SMN2 as well as data on the presence of the two-copy haplotype.

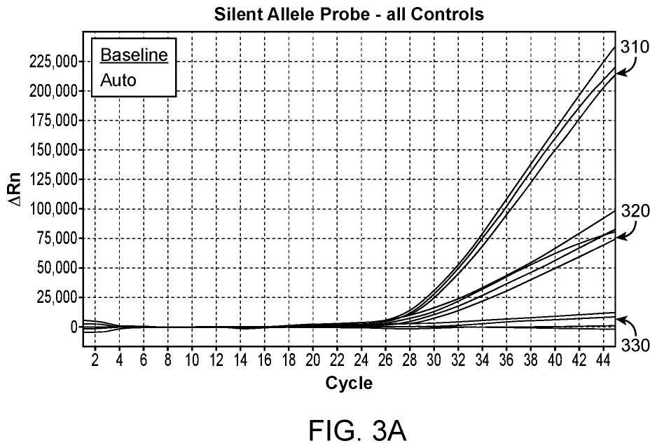

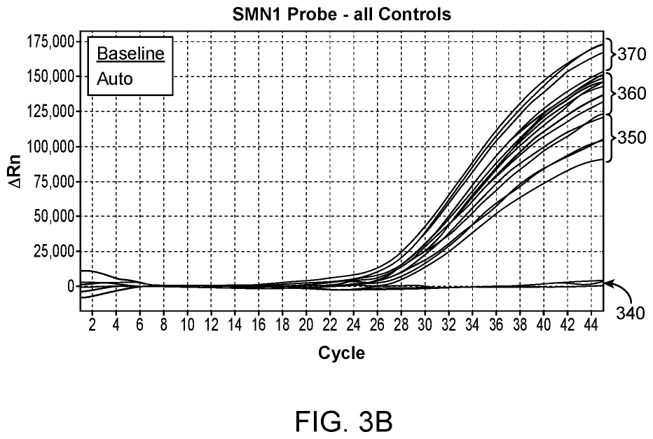

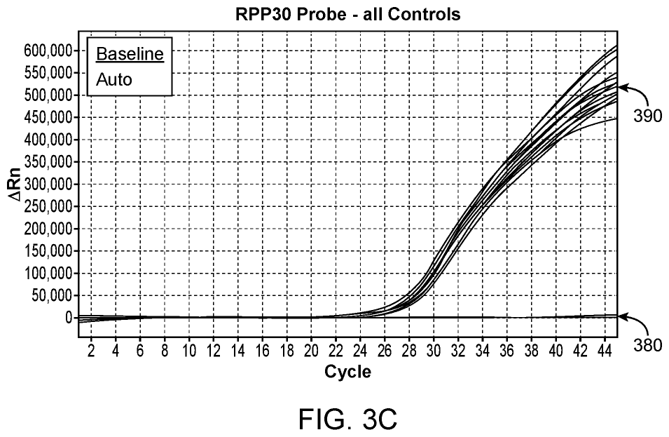

As described above, a qPCR reaction is run and data is collected regarding the signal generated from each probe. A software or processor may analyze data and identify the subject as having SMA or being a carrier or SMA. The signal from each probe is also monitored and graphed. FIG. 3A depicts an example of data that is generated the assay that is run on samples with a different copy number of the two-copy haplotype. The PCR cycle is denoted on the x-axis, while the signal generated from the probe is denoted on the y-axis. The probe signal that is recorded in this graph has affinity to the sequence that corresponds to the two-copy haplotype. Curves 310 demonstrate curves of a nucleic acid sample containing no two-copy haplotype. As such there is low signal to no signal even at cycle number 45. Curves 320 demonstrate curves of a nucleic acid samples containing one copy of the two-copy haplotype. As such, the signal begins increasing at approximately cycle 25 and at cycle 45 has reached a level of approximately 75,000 ΔRn. Curves 330 demonstrate curves of a nucleic acid samples containing two copies of the two-copy haplotype. As such, the signal begins increasing at approximately cycle 25 and at cycle 45 has reached a level of approximately 200,000 ΔRn. Between cycles 25 and 45, the samples containing 2 copies of the two-copy haplotype produce more signal than the samples containing only one copy of the two-copy haplotype, and the samples containing only one copy of the allele produce more signal than the samples that contain no copies of the two-copy haplotype. As such it is possible to determine the number of two-copy haplotypes in these samples.

FIG. 3B depicts an example of data that is generated the assay by running on samples comprising a different copy number of SMN1. The PCR cycle is denoted on the x-axis, while the signal generated from the probe is denoted on the y axis. The probe signal that is recorded in this graph has affinity to the sequence of the SMN1 gene. Curves 340 demonstrate curves of a nucleic acid samples containing no SMN1 gene. As such there is low signal to no signal even after cycle number 45. Curves 350 demonstrate curves of a nucleic acid samples containing one SMN1 gene. As such, the signal begins increasing at approximately cycle 25 and at cycle 45 has reached a level of approximately 100,000 ΔRn. Curves 360 demonstrate curves of a nucleic acid samples containing two copies of the SMN1 gene. As such, the signal begins increasing at approximately cycle 25 and at cycle 45 has reached a level of approximately 135,000 ΔRn. Curves 370 demonstrate curves of a nucleic acid samples containing three copies of the SMN1 gene. As such, the signal begins increasing at approximately cycle 25 and at cycle 45 has reached a level of approximately 165,000 ΔRn. Between cycles 25 and 45, the samples containing a larger number of copies of SMN1 generate more signal than samples that contain a smaller number of copies of SMN1 in these samples. As such it is possible to determine the number of SMN1 genes in the sample. Probes with affinity to SMN2 may also result in a similar graph.

To increase accuracy in copy number determination, a control probe can additionally be used as a reference to normalize the signal. FIG. 3C depicts an example of data that is generated by the assay by running on samples comprising a different copy number of SMN1 genes and two-copy haplotype. The probe signal that is recorded in this graph has affinity to the sequence of the SMN1 gene. Curve 380 demonstrates curves of nucleic acid samples containing no nucleic acids. As such there is no signal. Curve 390 demonstrates curves of nucleic acid samples containing two copies of RPP30 with various numbers of copies of the SMN1 gene and the two-copy haplotype. As demonstrated, the RPP30 signal is similar for all curves despite the samples containing various numbers of copies of the SMN1 gene and the two-copy haplotype. The signals obtained by the probe to SMN1 or SMN2 can be referenced using the RPP30 signal of the same sample to calculate the copy number.

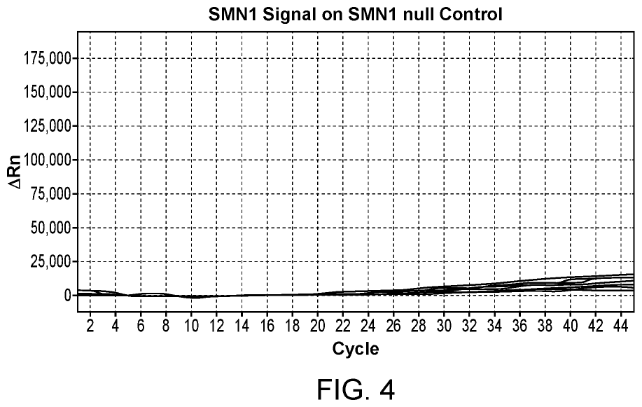

FIG. 4 depicts an example of data that is generated by the assay by running on samples that do not have SMN1 genes but do have SMN2 genes. The x-axis represents a general signal intensity of the SMN1 gene copy number probe. The probe signal that is recorded in this graph has affinity to the sequence of the SMN1 gene. Despite the similarity between SMN1 and SMN2, the signal intensity of the SMN1 gene copy number probe continues to be about 0-15,000 ΔRn at cycle 45. Compared to data in FIG. 3B, this demonstrates that there is little or no binding of the SMN1 gene copy number probe to a non SMN1 target.

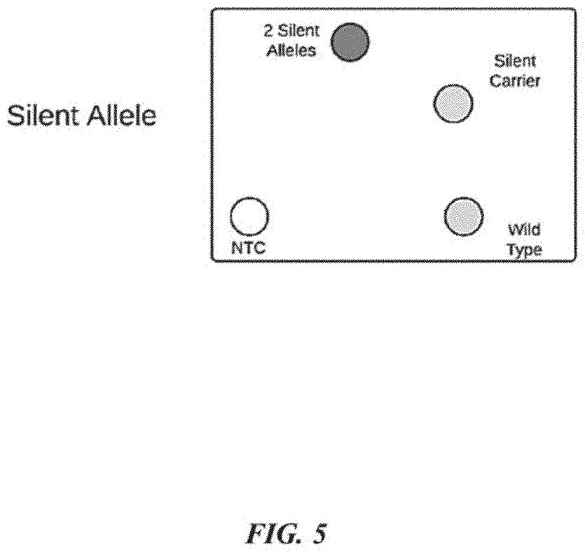

The data may be analyzed using allele discrimination plots to detect SMA and SMA carriers. FIG. 5 illustrates example general locations on the plots for specific genotypes of possible samples that may be used. The x-axis represents a general signal intensity correlated to the SMN1 and SMN2 gene copy number probes (related to detection of Amplicon 2). The y-axis represents a general signal intensity of probes correlated to the two-copy haplotype (related to detection of Amplicon 3). The white circle labeled NTC is a non-template control. This control sample has no template DNA and as such there is little or no signal generated for either the SMN1 gene copy number probe or the two-copy haplotype probe. The tan circle labeled Wild Type represents a wild-type sample in which the sample, for example, contains two SMN1 genes, one on each chromosome, and no copy of the two-copy haplotype allele. A healthy individual who is not a SMA carrier is represented by this circle. The signal intensity from the SMN1 and SMN2 gene copy number probe is higher than that of the non template control. The pink circle labeled Silent Carrier represents an individual who is a carrier for SMA and whose progeny may have SMA. This individual may have gene copy number of SMN1 and SMN2 that corresponds with a healthy individual but also have a two-copy haplotype. The signal intensity from the SMN1 and SMN2 gene copy number is higher than that of the non-template control. The signal intensity for the two-copy haplotype probe is also higher than that of the non-template control representing the presence of the two-copy haplotype. The red circle labeled 2 Silent Alleles represents a sample with two copies of the two-copy haplotype. The signal intensity for the two-copy haplotype probe is higher than the signal intensity of the two-copy haplotype probe for the Silent Carrier.

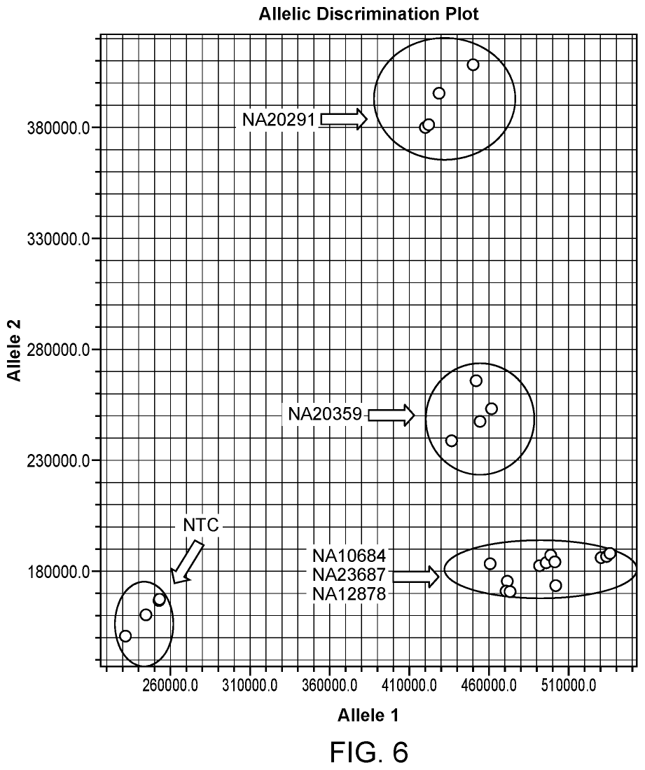

FIG. 6 depicts data point recorded for samples containing two-copy haplotypes. The x-axis represents a general signal intensity of the SMN1 and SMN2 gene copy number probe. The y-axis represents a general signal intensity of the two-copy haplotype probe. The green dots encircled and labeled “NTC” are non-template control samples, and have no signal for the two-copy haplotype or for SMN1. The red dot encircled and labeled “NA10684, NA23687, NA12878” are samples with no two-copy haplotype. These samples have higher signal intensity on the x-axis than the NTC owing to the presence of SMN1 and SMN2 genes in the sample, but a low signal from the two-copy haplotype probe. The green dots encircled and labeled “NA20359” are samples with one copy of the two-copy haplotype. These samples have higher signal intensity on the x- and y-axes than the NTC owing to the presence of SMN1 and SMN2 genes in the sample and a higher signal from the two-copy haplotype probe. The blue dots encircled and labeled “NA20291” are samples with two copies of the two-copy haplotype. These samples have higher signal intensity on the x-axis than the NTC owing to the presence of SMN1 and SMN2 genes in the sample, and a higher signal intensity on the y-axis from the two-copy haplotype probe. As the “NA20291” sample contains two copies of the two-copy haplotype signal intensity from the two-copy haplotype probe is even higher for the “NA20359” sample containing only one copy of the two-copy haplotype.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-23. (canceled)

24. A method for identifying a genetic signature(s) associated with spinal muscular atrophy (SMA) in a nucleic acid sample of a subject, comprising:
(a) in a single vessel, providing a reaction mixture comprising said nucleic acid sample of said subject, a polymerizing enzyme and a probe set, which probe set comprises (i) a first probe that has sequence specificity for an SMN1 gene at a first locus of said nucleic acid sample, (ii) a second probe that has sequence specificity for an SMN2 gene at said first locus, (iii) a third probe that has sequence specificity for said SMN1 or SMN2 gene at a second locus of said nucleic acid sample, which second locus is different than said first locus, and (iv) a fourth probe that has sequence specificity for a genetic aberration of said SMN1 gene at said second locus;
(b) subjecting said reaction mixture in said single vessel to conditions sufficient to generate a plurality of amplicons corresponding to said first locus and said second locus;
(c) detecting said plurality of amplicons; and
(d) based at least in part on said plurality of amplicons detected in (c), (i) identify said genetic signature(s) associated with SMA, with an accuracy of at least 90%.

25. The method of claim 24, wherein said genetic aberration of said SMN1 gene is a two-copy haplotype.

26. The method of claim 24, wherein (d) comprises identifying (i) a copy number in SMN1 or (ii) said genetic aberration of said SMN1 gene.

27. The method of claim 26, wherein (d) comprises identifying (i) a copy number in SMN1 and (ii) said genetic aberration of said SMN1 gene.

28. The method of claim 24, wherein (c) comprises measuring a plurality of intensities corresponding to said first probe, second probe, third probe and fourth probe.

29. The method of claim 28, further comprising measuring said plurality of intensities against an intensity from a control probe.

30. The method of claim 24, wherein (b) comprises performing a polymerase chain reaction on said nucleic acid sample at said first locus and said second locus.

31. The method of claim 30, wherein said reaction mixture comprises primers targeting said first locus and said second locus.

32. The method of claim 24, wherein said nucleic acid sample is a chromosome or a derivative of said chromosome.

33. The method of claim 24, wherein said nucleic acid sample is obtained from said subject and provided in said single vessel without any filtration, extraction or purification.

34. The method of claim 24, wherein said accuracy is at least 95%.

35. The method of claim 34, wherein said accuracy is at least 98%.

36. The method of claim 24, wherein said detecting comprises detecting optical signals corresponding to said plurality of amplicons.

37. The method of claim 36, wherein said optical signals are fluorescent signals.

38. A system for identifying a genetic signature(s) associated with SMA in a nucleic acid sample of a subject, comprising:
a single vessel configured to contain a reaction mixture comprising said nucleic acid sample of said subject, a polymerizing enzyme and a probe set, which probe set comprises (i) a first probe that has sequence specificity for an SMN1 gene at a first locus of said nucleic acid sample, (ii) a second probe that has sequence specificity for an SMN2 gene at said first locus, (iii) a third probe that has sequence specificity for said SMN1 or SMN2 gene at a second locus of said nucleic acid sample, which second locus is different than said first locus, and (iv) a fourth probe that has sequence specificity for a genetic aberration of said SMN1 gene at said second locus;
a detector operatively coupled to said single vessel; and
one or more computer processors operatively coupled to said single vessel, wherein said one or more computer processors are individually or collectively programmed to (i) subject said reaction mixture in said single vessel to conditions sufficient to generate a plurality of amplicons corresponding to said first locus and said second locus; (ii) use said detector to detect said plurality of amplicons; and (iii) based at least in part on said plurality of amplicons detected in (ii), identify a genetic signature(s) associated with SMA with an accuracy of at least 90%.

39. The system of claim 38, wherein said genetic aberration of said SMN1 gene is a two-copy haplotype.

40. The system of claim 38, wherein said one or more computer processors are individually or collectively programmed to identify (i) a copy number in SMN1 or (ii) said genetic aberration of said SMN1 gene.

41. The system of claim 40, wherein said one or more computer processors are individually or collectively programmed to identify (i) a copy number in SMN1 and (ii) said genetic aberration of said SMN1 gene.

42. The system of claim 38, wherein said detector is an optical detector.

43. The system of claim 38, further comprising a heating unit in thermal communication with said single vessel, wherein said one or more computer processors are individually or collectively programmed to direct said heating unit to subject said reaction mixture to one or more heating and cooling cycles to generate said plurality of amplicons.

44. The system of claim 38, further comprising a heating unit in thermal communication with said single vessel, wherein said one or more computer processors are individually or collectively programmed to direct said heating unit to subject said reaction mixture to heating to generate said plurality of amplicons.

45. The system of claim 44, wherein said heating is isothermal heating.
